(19) United States
(12) Patent Application Publication
(10) Pub. No.: US 2026/0315099 A1
(43) Pub. Date: Oct. 15, 2026

(54) ENDOSCOPIC FULL THICKNESS RESECTION (EFTR) DEVICE

(71) Applicant: United States Endoscopy Group, Inc., Mentor, OH (US)

(72) Inventors: Jessica Russo, Sagamore Hills, OH (US); Gary Mann, Painesville, OH (US); Seth Byers, Madison, OH (US); Sven Huijs, Madison, OH (US); Eugene Skelton, Dublin (IE); Anthony Wright, Waterford (IE)

(21) Appl. No.: 19/482,047

(22) PCT Filed: May 15, 2024
(86) PCT No.: PCT/US2024/029377

§ 371 Date: Nov. 6, 2025

Related U.S. Application Data
(60) Provisional application No. 63/503,301, filed on May 19, 2023.

(51) Int. Cl. A61B 17/3205; A61B 1/00; A61B 17/00

(52) U.S. Cl. CPC A61B 17/32056; A61B 1/00131; A61B 17/00234; A61B 2017/00269; A61B 2017/00296; A61B 2017/00353

(57) ABSTRACT

A device for closing and cutting tissue for use with an endoscope. The device includes a distal assembly for closing and cutting tissue and a proximal assembly for actuating the distal assembly. The proximal and distal assemblies may be coupled with a catheter sheath assembly. The distal assembly may deploy a cinch element and cut cinched tissue proximally to the deployed cinch element. The proximal assembly may deploy and actuate the distal assembly, such as via a single stroke.

### CLAIM OF PRIORITY AND CROSS-REFERENCE TO RELATED APPLICATIONS

This application claims priority to U.S. Provisional Patent Application Ser. No. 63/503,301, filed on May 19, 2023, entitled “ENDOSCOPIC FULL THICKNESS RESECTION (EFTR) DEVICE,” which is incorporated by reference herein in its entirety.

### FIELD OF THE INVENTION

The various embodiments relate to a closure and retrieval device to be used with an endoscope, and, more particularly, to an endoscopic device with a closure and retrieval assembly configured to ligate and retrieve tissue and actuation assembly configured to actuate the closure and retrieval assembly. The closure and retrieval assembly uses a cinch element to surround tissue to be ligated and a cutting element to cut the cinched tissue for retrieval. The actuation assembly is configured to actuate the cinch element and the cutting mechanism in succession. The device can be used to cut off blood supply to tissue and to cut and remove tissue but is not limited to such uses.

### BACKGROUND

Endoscopes are known in the medical arts and are used for numerous medical procedures. One such procedure is removing targeted tissue from a human subject, such as targeted tissue from the gastro-intestinal mucosal wall of a human subject. Various types of tissue, such as for example, polyps, lesions, tumors, or adenomas, may be removed for various medical purposes, such as for treatment or diagnostic testing.

Mechanical devices, such as snares, may be used to separate a polyp or growths of tissue from the gastrointestinal wall of a patient. Specifically, an operator may deploy or open a snare loop and position the snare around a polyp. By retrieval or closure of the snare loop, the operator can cut into the tissue and separate the tissue from the gastrointestinal wall. Subsequently, the operator may use a suction source to capture and retain the tissue after separation from the wall.

One such procedure is removing targeted muscularis from the gastrointestinal (GI) tract to remove dysplasia that has invaded past the mucosal layer of tissue. In some instances, such as where dysplasia has invaded past multiple layers of tissue, a clip or cinch element may be deployed around the targeted tissue to assist in the healing process after the targeted tissue has been separated. Further, some polypectomy techniques may use cauterization to assist in removing the targeted tissue. For example, a cauterizing device may heat the targeted tissue from the gastrointestinal wall to assist in the removal of the targeted tissue.

### SUMMARY

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. The description herein relates to systems, assemblies, methods, devices, apparatuses, combinations, etc. that may be utilized for cutting and removing tissue, such as dysplasia that has invaded past the mucosal layer of tissue in the gastrointestinal tract. Various features and steps as described elsewhere in this disclosure may be included in the examples summarized here. Further, the treatment techniques, methods, operations, steps, etc. described or suggested herein can be performed on a living animal or on a non-living simulation, such as on a cadaver, simulator (e.g., with the body parts, tissue, etc. being simulated), etc.

In an exemplary embodiment, a closure and retrieval assembly for an endoscopic device is provided. The closure and retrieval assembly includes a housing having an outer surface and an inner surface defining a tissue receiving portion and a collar having an inner surface and an outer surface. The inner surface of the collar is configured to fit around the outer surface of the housing. The closure and retrieval assembly also include a cinch element movable between an open position configured to fit around the outer surface of the housing and a closed position configured to cinch tissue recruited into the tissue receiving portion. The closure and retrieval assembly further includes a cutting element coupled with the collar. The closure and retrieval assembly is movable between an undeployed configuration and a deployed configuration. The collar and the cinch element are disposed around the outer surface of the housing when the assembly is in the undeployed configuration. The cinch element and the collar are disposed distally relative to the housing when the assembly is in the deployed configuration.

In an exemplary embodiment, an actuation assembly for an endoscopic device is provided. The actuation assembly includes a shaft having a slider coupling portion and a connector coupling portion, a slider having a coupler configured to operatively couple with the slider coupling portion of the shaft, a connector having a projection configured to operative couple with the connector coupling portion of the shaft, and a spool configured to slide along the shaft and configured to operatively couple with the connector and the slider. The actuation assembly is movable between an unactuated configuration with the connector and the spool disposed near a distal end of the shaft, a partially actuated configuration, and a fully actuated configuration with the spool and the slider disposed near a proximal end of the shaft. The connector is coupled with the spool and the slider is coupled with the shaft when the assembly is moved from the unactuated configuration to the partially actuated configuration. The slider is coupled with the spool and the connector is coupled with the shaft when the assembly is moved from the partially actuated configuration to the fully actuated configuration.

In an exemplary embodiment, a closure and retrieval device is provided. The closure and retrieval device includes a closure and retrieval assembly movable between an undeployed configuration and a deployed configuration, an actuation assembly movable between an unactuated configuration and an actuated configuration, and a catheter sheath assembly configured to operatively couple the closure and retrieval assembly and the actuation assembly. The closure and retrieval assembly includes a housing having an outer surface and an inner surface defining a tissue receiving portion, a collar including a cutting element, the collar being disposed around the outer surface of the housing in the undeployed configuration, and a cinch element disposed around the outer surface of the collar and distal to the collar in the undeployed configuration. The actuation assembly includes a shaft extending between a proximal end and a distal end, a connector proximally slidable relative to the shaft in the undeployed configuration and coupled to the shaft in the actuated configuration, a slider coupled to the shaft in the unactuated configuration and proximally slidable relative to the shaft in the actuated configuration, and a spool configured to slide along the shaft and configured to operatively couple with the connector and the slider. Distal movement of the shaft relative to the spool is configured to move the connector proximally relative to the shaft until the connector couples with the shaft and the spool decouples the slider from the shaft such that the slider is slidable proximally relative to the shaft. Actuation of the actuation assembly is configured to move the closure and retrieval assembly from the undeployed configuration to the deployed configuration. The cinch element is moved distally from the housing when the closure and retrieval assembly is moved from the undeployed configuration to the deployed configuration.

### BRIEF DESCRIPTION OF THE DRAWINGS

To further clarify various aspects of implementations of the present disclosure, a more particular description of the certain examples and implementations will be made by reference to various aspects of the appended drawings. These drawings depict only example implementations of the present disclosure and are therefore not to be considered limiting of the scope of the disclosure. Moreover, while the Figures can be drawn to scale for some examples, the Figures are not necessarily drawn to scale for all examples. Examples and other features and advantages of the present disclosure will be described and explained with additional specificity and detail through the use of the accompanying drawings in which:

FIG. 1 is a side view of a closure and retrieval device in accordance with an embodiment of the present invention;

FIGS. 2 and 3 are perspective views of a closure and retrieval assembly in accordance with an embodiment of the present invention for use with the closure and retrieval device of FIG. 1, the closure and retrieval assembly having a housing, a collar, and a cinch element;

FIGS. 4-6 are various views of a housing in accordance with an embodiment of the present invention for use with the closure and retrieval assembly of FIGS. 2-3;

FIG. 7 is a view of a collar in accordance with an embodiment of the present invention for use with the closure and retrieval assembly of FIGS. 2-3;

FIG. 8 is a perspective view of the collar of FIG. 7 disposed on the housing of FIGS. 4-6;

FIGS. 9 and 10 are various views of a cinch element in accordance with an embodiment of the present invention for use with the closure and retrieval assembly of FIGS. 2-3, the cinch element disposed in an open position;

FIGS. 11 and 12 are various views of the cinch element of FIGS. 9-10 in a closed position;

FIG. 13 is a side cross-sectional view of the cinch element of FIG. 11 taken along line A-A of FIG. 11;

FIGS. 14-17 are schematic illustrations of a closure and retrieval assembly in various stages of deployment and actuation;

FIGS. 18-20 are various views of a housing in accordance with an embodiment of the present invention, the housing including a coupling portion and a receiving portion;

FIG. 21 is a side cross-sectional view of the coupling portion of FIGS. 18-20 taken along line A-A of FIG. 20;

FIGS. 22 and 23 are perspective views of the receiving portion of FIGS. 18-20;

FIGS. 24-27 are various views of a collar in accordance with an embodiment of the present invention;

FIGS. 28-29 are perspective views of the collar of FIGS. 24-27 coupled with a cutting element;

FIGS. 30 and 31 are various views of the collar and the cutting element of FIGS. 28-29 disposed around the housing of FIGS. 18-20 in an undeployed configuration;

FIG. 32 is a top view of the collar, the cutting element, and the housing of FIGS. 30-31 in a deployed configuration;

FIG. 33 is a top view of the collar and the cutting element of FIG. 32 with the collar removed;

FIG. 34 is a side view of a closure and retrieval assembly in accordance with an embodiment of the present invention;

FIGS. 35-45B are schematic illustrations of a collar and a cutting embodiment in accordance with various embodiments of the present invention;

FIGS. 46A-46D illustrate schematic views of a cutting element of a closure and retrieval assembly in accordance with another embodiment of the present invention;

FIG. 47A is a side view of a closure and retrieval assembly in accordance with various embodiments of the present invention, the closure and retrieval assembly including a safety cap;

FIG. 47B is a side view of a closure and retrieval assembly in accordance with various embodiments of the present invention, the closure and retrieval assembly including another safety cap;

FIGS. 47C-47E are various views of the safety cap of FIG. 47B;

FIG. 47F is a side cross-sectional view of the safety cap of FIGS. 47B-47E;

FIGS. 48-50 are perspective views of an actuation assembly in accordance with an embodiment of the present invention in various positions of actuation;

FIGS. 51-53 are side views of an actuation assembly in accordance with an embodiment of the present invention with half of a spool removed for illustrative purposes;

FIGS. 54A and 54B are perspective views of a shaft of the actuation assemblies of FIG. 48-53 in accordance with an embodiment of the present invention;

FIGS. 55A and 55B are perspective views of a connector in accordance with an embodiment of the present invention, the spool being operable with the actuation assemblies of FIGS. 48-53;

FIG. 55C is a side cross-sectional view of the connector of FIGS. 55A and 55B taken along line A-A of FIG. 55A;

FIG. 55D is a top perspective cross-sectional view of the connector of FIGS. 55A and 55B taken along line B-B of FIG. 55A;

FIGS. 56A-56C are various views of a slider in accordance with an embodiment of the invention, the slider being operable with the actuation assemblies of FIGS. 48-53;

FIG. 57 is a perspective view of the slider of FIG. 56A-56C coupled with the shaft of FIGS. 54A-54B;

FIGS. 58A-58C are various views of an upper portion of the slider of FIGS. 56A-56C in accordance with an embodiment of the invention;

FIGS. 59A-59C are various views of a lower portion of the slider of FIGS. 56A-56C in accordance with an embodiment of the invention;

FIGS. 60A-60B are various views of a spool in accordance with an embodiment of the invention, the spool being operable with the actuation assemblies of FIGS. 48-53;

FIGS. 61A-61B are various views of a spool half of the spool of FIGS. 60A-60B;

FIG. 62 is a side view of an actuation assembly in accordance with another embodiment of the present invention coupled with an energy source;

FIG. 63 is a side view of an actuation assembly in accordance with another embodiment of the invention, the actuation assembly including a stopper;

FIG. 64 is a perspective view of the stopper of FIG. 63;

FIG. 65 is a perspective view of the stopper of FIG. 63 coupled with the shaft of FIG. 63;

FIG. 66-68 are various views of the actuation assembly of FIGS. 51-53 coupled with a catheter sheath assembly, the actuation assembly being in the unactuated position;

FIG. 69 is a side view of the closure and retrieval assembly of FIG. 34 coupled with the catheter sheath assembly;

FIG. 70 is a side cross sectional view of the closure and retrieval assembly of FIG. 69;

FIGS. 71-72 are various views of the actuation assembly and catheter sheath assembly of FIGS. 66-68 in a partially actuated position;

FIG. 73 is a side view of the closure and retrieval assembly and catheter sheath assembly of FIG. 69 in a deployed configuration; and

FIGS. 74-75 are various views of the actuation assembly and catheter sheath assembly of FIGS. 66-68 in a fully actuated configuration.

### DETAILED DESCRIPTION

The following description refers to the accompanying drawings, which illustrate specific embodiments of the present disclosures, and describes exemplary embodiments in accordance with the general inventive concepts and is not intended to limit the scope of the invention or the claims in any way. Indeed, the invention as described by the claims is broader than and not limited by the exemplary embodiments set forth herein, and the terms used in the claims have their full ordinary meaning.

The general inventive concepts will now be described with occasional reference to the exemplary embodiments. Example embodiments of the present disclosure are directed to devices and methods for cutting and removing tissue. Various embodiments of devices and systems for cutting and removing tissue are disclosed herein, and any combination of these options can be made unless specifically excluded. In other words, individual components of the disclosed devices and systems can be combined unless mutually exclusive or otherwise physically impossible.

Unless otherwise defined, all technical and scientific terms used herein have the same meaning as commonly understood by one of ordinary skill in the art encompassing the general inventive concepts. The terminology set forth in this detailed description is for describing particular embodiments only and is not intended to be limiting of the general inventive concepts. As used in this detailed description and the appended claims, the singular forms “a,” “an,” and “the” are intended to include the plural forms as well, unless the context clearly indicates otherwise.

As described herein, when one or more components are described as being connected, joined, affixed, coupled, attached, or otherwise interconnected, such interconnection can be direct as between the components or can be indirect such as through the use of one or more intermediary components. Also, as described herein, reference to a “member,” “component,” or “portion” shall not be limited to a single structural member, component, or element, but can include an assembly of components, members, or elements.

Unless otherwise indicated, all numbers, such as for example, numbers expressing measurements or physical characteristics, used in the specification and claims are to be understood as being modified in all instances by the term “about.” Accordingly, unless otherwise indicated, the numerical properties set forth in the specification and claims are approximations that may vary depending on the suitable properties sought to be obtained in embodiments of the invention. Notwithstanding that the numerical ranges and parameters setting forth the broad scope of the general inventive concepts are approximations, the numerical values set forth in the specific examples are reported as precisely as possible. Any numerical values, however, inherently contain certain errors necessarily resulting from error found in their respective measurements. Also, as described herein, the terms “substantially” and “about” are defined as at least close to (and includes) a given value or state (preferably within 10% of, more preferably within 1% of, and most preferably within 0.1% of).

In discussing the invention, the terms “proximal” and “distal” are often used. These terms are used to describe a position or direction with reference to the operator of the device. For example, the proximal position or proximal direction is toward the user or operator of the device, and the distal position or distal direction is away from the user or operator of the device, i.e., position or direction toward the endoscope.

When generally discussing the invention and embodiments of the invention, the terms “portrait” and “landscape” will be used to describe an orientation of an object. The term “portrait” and the term “landscape” have their common ordinary meaning, and are used in the same manner in which one would use each term to describe the orientation of a common and well-known object or thing, such as for example, a piece of paper or a picture frame.

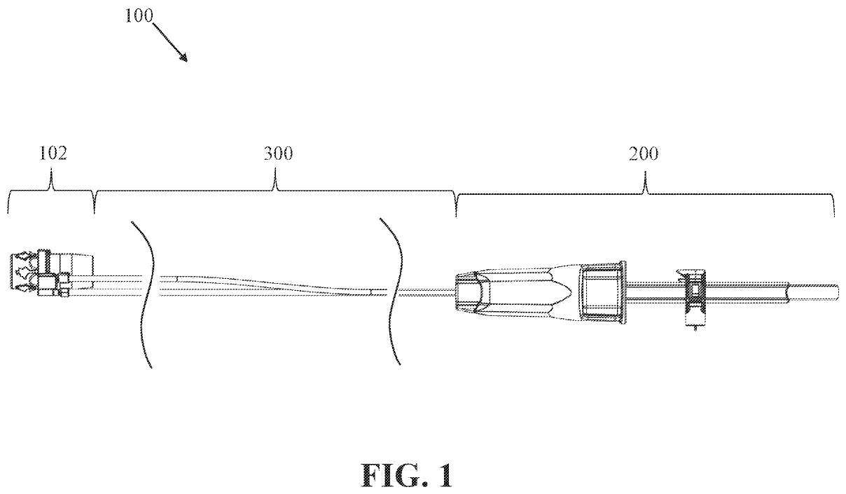

An endoscopic closure and retrieval device 100 for cinching target tissue and retrieving or otherwise extracting the target tissue within a subject is illustrated in FIG. 1. The device 100 may be disposed through or around a distal end of an endoscope and may be operated by a user at a proximal end of the endoscope via one or more catheters or sheaths extending through a channel located within and extending through an endoscope. The device 100 is constructed and configured such that it may be inserted into a subject through an orifice or small incision and operated to cinch tissue and cut the cinched tissue, such as for retrieval or extraction from the subject. The device 100 may be configured to cinch and cut a full thickness of target tissue, such as through all four layers of tissue of the GI tract, to remove the targeted tissue, such as an entire dysplasia that has invaded past the mucosal tissue layer.

The device 100 includes a closure and retrieval assembly 102 which may form or otherwise couple to a distal end of the endoscope. The closure and retrieval assembly 102 is configured to cinch and cut target tissue. The closure and retrieval assembly 102 may be configured and assembled to cinch the target tissue before the target tissue is cut. For example, the closure and retrieval assembly 102 may be configured and assembled such that the target tissue is not cut before the tissue is cinched.

The device 100 also includes an actuation assembly 200 which is operable by a user and configured to actuate the device 100. The actuation assembly 200 may be configured and coupled to the remainder of the device 100, such as to the closure and retrieval assembly 102, to actuate the device 100 in a single motion. For example, a single stroke of the actuation assembly 200 may cause the closure and retrieval assembly 102 to cinch and cut tissue.

The device 100 can be used with any suitable or conventional endoscopic or laparoscopic surgical equipment. For purposes of this disclosure, the device 100 is described in the context of use with an endoscope/colonoscope/sigmoidoscope type apparatus (not illustrated), of conventional or suitable construction. The scope is provided with an elongated body having a controllably flexible projecting end region. Surgical instruments, such as the device 100, may be introduced around the scope body for retrieving targeted tissue by the surgeon manipulating the scope. Additionally or alternatively, surgical instruments, such as the device 100, may be introduced through an instrument channel, which extends through the scope body, for retrieving tissue targeted by the surgeon manipulating the scope.

Referring to FIG. 1, an endoscopic device 100 is illustrated. The device 100 is for use with an endoscope (not shown) and includes a closure and retrieval assembly 102 at a distal end, an actuation assembly 200 at a proximal end, and a catheter sheath assembly 300 disposed between the closure and retrieval assembly 102 and the actuation assembly 200. The closure and retrieval assembly 102 is configured to cinch and cut tissue, such as dysplasia that has invaded into the muscularis of the GI tract. The closure and retrieval assembly 102 may also be operable to retrieve the severed tissue. The actuation assembly 200 is configured to control deployment and actuation of the closure and retrieval assembly 102.

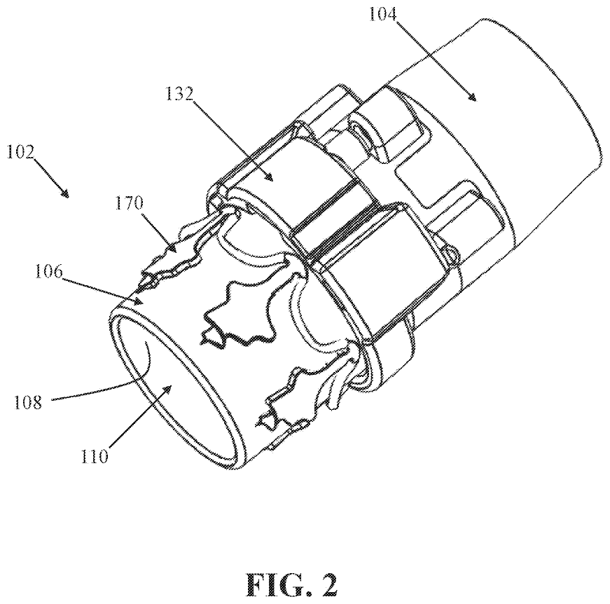

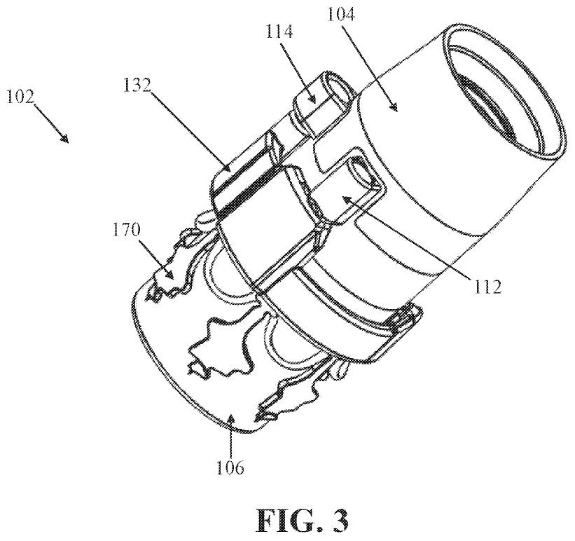

Referring now to FIGS. 2-3, the closure and retrieval assembly 102 includes a housing 104 configured to couple with a distal tip or end of an endoscope (not shown), a cinch element 170 configured to cinch tissue, and a collar 132 operably disposed around the housing 104. The collar 132 may include or be coupled with a cutting element operably disposed within the collar 132. The cinch element 170 and the collar 132 are operably deployed from the housing 104 to cinch and cut, respectively, tissue that has been recruited into the housing 104. It will be understood that the cinching of the closure and retrieval assembly 102 encompasses clipping, ligating, or other means of securing tissue and the cutting of the closure and retrieval assembly 102 encompasses severing, ablating, cauterizing, cold-cutting, or other means for separating tissue.

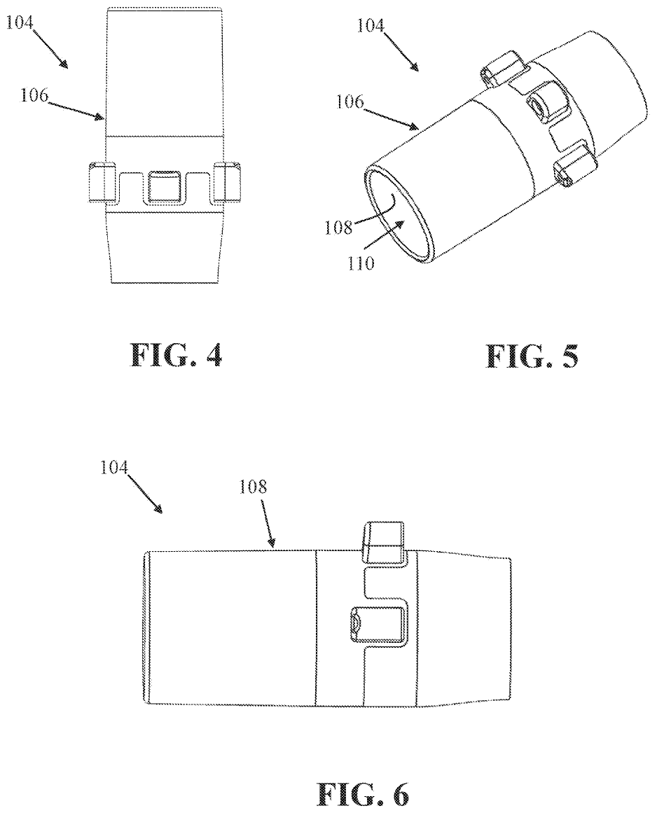

As shown in FIGS. 4-6, the housing 104 may be a substantially hollow cylinder with an outer surface 106 and an inner surface 108. The inner surface 108 may define a tissue receiving portion 110 configured to receive target tissue. The housing 104 may be sized, shaped, and configured such that the tissue receiving portion 110 is large enough to receive the targeted tissue, such as all four layers of target tissue in a GI tract. The housing 104 may be coupled with the actuation assembly 200 via the catheter sheath assembly 300 to control the deployment, position, or configuration of the closure and retrieval assembly 102, such as detailed below.

The housing 104 may be sized, shaped, and configured to couple with the distal tip of the endoscope, such as with the proximal portion of the housing 104 disposed around the distal tip of the endoscope. A proximal end or portion of the housing 104 may be tapered radially inwardly to fit the distal tip of the endoscope. The inner surface 108 of the housing 104 may include ribs, dimples, divots, threads, grooves, textured surfaces, or the like near the proximal end to engage with the outer surface of the distal end of the endoscope. In addition or in the alternative, the housing 104 may be coupled to the distal tip of the endoscope via adhesives, welding, over-molding, press-fitting, or the like. While the housing 104 has been described as having a proximal portion configured to couple with the distal end of an endoscope, it will be understood that the housing 104 may be coupled to the distal end of an endoscope via a separate component or connector, such as a tapered connector with threads, dimples, divots, ribs, grooves, or textured surfaces configured to couple the housing 104 to the endoscope.

The device 100 may include or be coupled with one or more means for recruiting or otherwise drawing tissue into the tissue receiving portion 110 of the housing 104. The means for recruiting tissue may extend through the endoscope, such as through channel located within and extending through an endoscope, and may extend into or through the tissue receiving portion 110 of the housing 104. In some embodiments, one or more grasping instruments, such as forceps, hooks, prongs, or the like, or any combination thereof, may be advanced distally through the endoscope and the housing 104 to grasp or secure tissue and then retracted proximally to recruit the tissue into the tissue receiving portion 110. The grasping instrument may be disposed through the catheter sheath assembly 300 and/or the endoscope. The grasping instrument may be coupled with the actuation assembly 200 or a separate controller such that a user may operate to control the grasping instrument. In other embodiments, suction, such as via a suction lumen of the endoscope, may be used to suck or otherwise draw tissue into the tissue receiving portion 110 from the distal end of the closure and retrieval assembly 102.

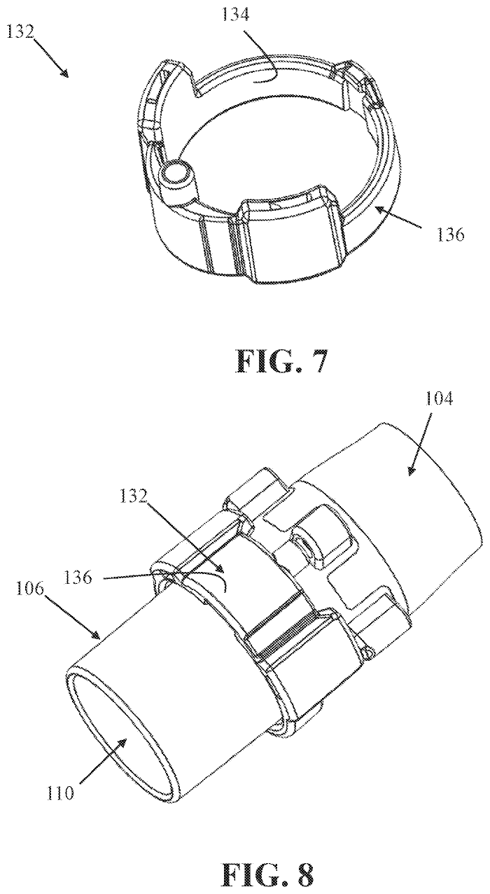

As shown in FIGS. 7-8, the collar 132 may be substantially ring-shaped with an inner surface 134 having an inner diameter and an outer surface 136 having an outer diameter. The inner surface 134 may be sized, shaped, and configured such that the collar 132 may be disposed around the outer surface 106 of the housing 104 when the closure and retrieval assembly 102 is in an undeployed configuration and such that the collar 132 may be moved distally beyond a distal end of the housing 104 when the closure and retrieval assembly 102 is moved to a deployed configuration, such as described below. For example, the inner diameter of the inner surface 134 of the collar 132 may be slightly larger than a diameter of the outer surface 106 of the housing 104 such that the collar 132 may slide along the outer surface 106 of the housing 104. The inner diameter of the inner surface 134 of the collar 132 may be less than a medial or proximal portion of the housing 104, such as a portion of the housing 104 coupled to the catheter sheath assembly 300, such that the collar 132 is prevented from sliding proximally past a proximal end of the housing 104. The collar 132 may be coupled with the actuation assembly 200 via one or more catheters, spring sheaths, actuations wires, or the like to control the deployment, position, or configuration of the collar 132 and the closure and retrieval assembly 102, such as described below.

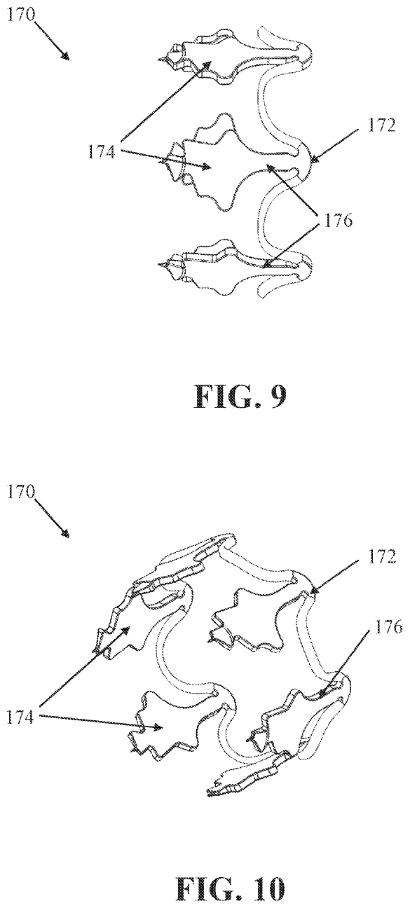

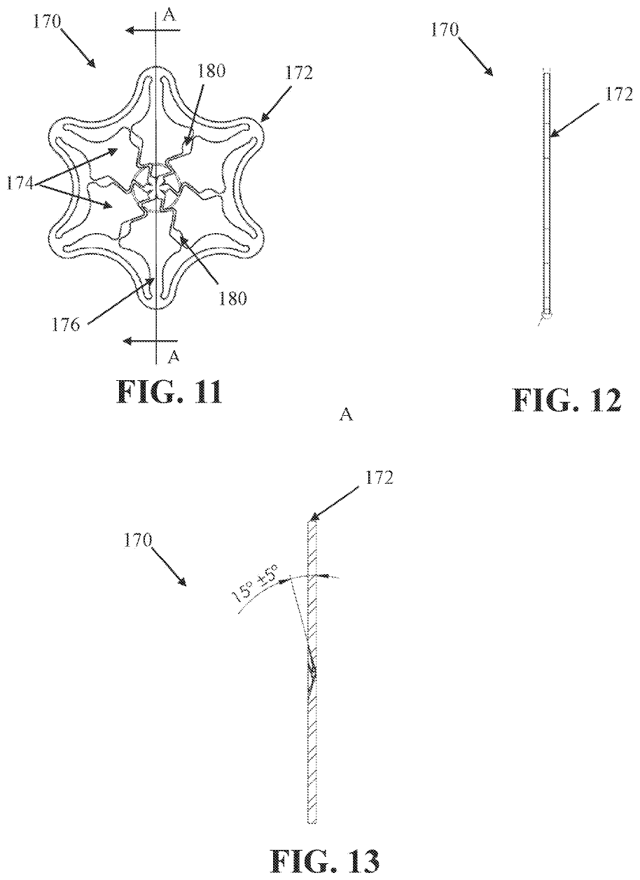

Referring now to FIGS. 9-13, the cinch element 170 may be configured to move from an open position (FIGS. 9-10) configured to slide along the outer surface 106 of the housing 104 to a closed position (FIGS. 11-13) configured to cinch or otherwise secure tissue, such as when the cinch element 170 is moved off the housing 104. In the illustrated embodiment, the cinch element 170 is substantially disk-shaped in the closed position. The cinch element 170 may include a substantially circular perimeter 172 having an outer diameter. The cinch element 170 may also include a plurality of projections or tines 174 extending radially inwardly from the perimeter 172 when the cinch element 170 is in the closed position. The tines 174 may be sized, shaped, and configured such that the tines 174 substantially close a medial portion of the cinch element 170 when the cinch element 170 is in the closed position. The cinch element 170 may be configured such that the tines 174 are biased toward the closed position. For example, the cinch element 170 may comprise a shape memory material, such as Nitinol.

As shown in FIG. 9-10, the cinch element 170 may be moved to an open position. In the open position, the tines 174 may extend distally (e.g., tangentially) from the perimeter 172 such that the medial portion of the cinch element 170 is relatively open, thereby defining an inner diameter of the perimeter 172. Tissue may be received in the medial portion of the cinch element 170 when the cinch element 170 is in the open position and the cinch element 170 may be moved to the closed position, thereby securing the tissue between the tines 174.

The cinch element 170 may be sized, shaped, and configured such that it may be slidably disposed around the outer surface 106 of the housing 104 in the open position. The cinch element 170 may be sized, shaped, and configured such that the inner diameter of the perimeter 172 of the cinch element 170 in the open position is larger than the outer diameter of the outer surface 106 of the housing 104. The outer surface 106 of the housing 104 may bias or otherwise maintain the tines 174 of the cinch element 170 into the open position while the cinch element 170 is disposed around the housing 104. The inner diameter of the perimeter 172 of the cinch element 170 may also be less than the outer diameter of the outer surface 136 of the collar 132 such that a distal end of the collar 132 may abut the cinch element 170 when the collar 132 and the cinch element 170 are disposed around the housing 104, thereby preventing the cinch element 170 from sliding past the collar 132. The abutment between the cinch element 170 and the collar 132 may also cause the cinch element 170 to move distally with distal movement of the collar 132 on the housing 104, as described below.

The cinch element 170 may move from the open position to the closed position when the tines 174 are no longer biased radially outwardly. For example, the cinch element 170 may move to the closed position when the cinch element 170 is moved off the outer surface 106 of the housing 104, such as when the housing 104 is properly positioned and tissue is recruited into the tissue receiving portion 110 such that the cinch element 170 cinches the recruited tissue. The cinch element 170 may be configured to remain cinched on the tissue long enough to allow the tissue to heal, such as after the recruited tissue has been cut. Additionally, the outer diameter of the cinch element 170 in the closed position may be larger than the inner diameter of the inner surface 134 of the collar 132 such that the collar 132 is prevented from moving distally past the deployed cinch element 170, as described below.

The closure and retrieval assembly 102 is configured to be positioned in a native body in a first or undeployed configuration (e.g., FIGS. 2-3) with the collar 132 disposed around a medial portion of the housing 104 and with the cinch element 170 disposed in the open position around a distal portion of the housing 104 between the collar 132 and the distal end of the housing 104. For example, the closure and retrieval assembly 102 may be inserted through an orifice or small incision on the distal end of an endoscope and then positioned above targeted tissue, such as tissue for retrieval or extraction from the subject.

The closure and retrieval assembly 102 may then be moved to a second or partially deployed configuration in which the collar 132 is moved distally relative to the housing 104 such that the cinch element 170 is moved off the distal end of the housing 104 and into the closed position to cinch target tissue. The closure and retrieval assembly 102 may then be moved to a third or fully deployed position in which the collar 132 with the cutting element 180 is moved distally beyond the distal end of the housing 104 such that the collar 132 is disposed between the deployed cinch element 170 and the distal end of the housing 104. The closure and retrieval assembly 102 may then be moved to a fourth or actuated configuration in which the cutting element 180 is actuated to cut the tissue recruited into the tissue receiving portion 110 proximally from the deployed cinch element 170.

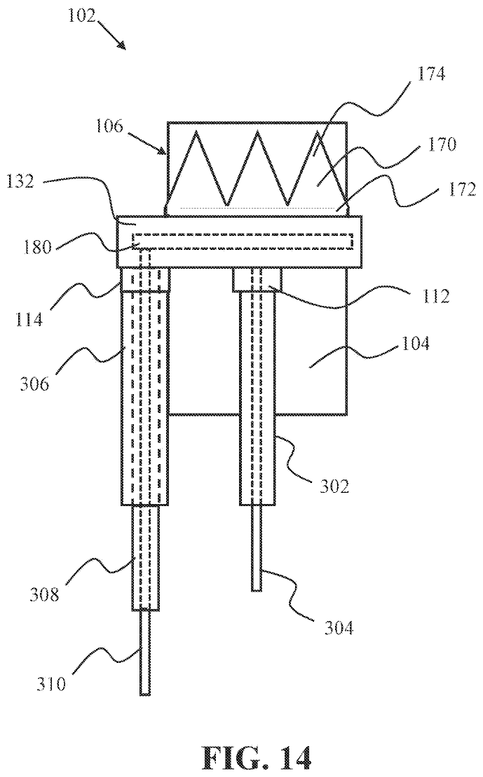

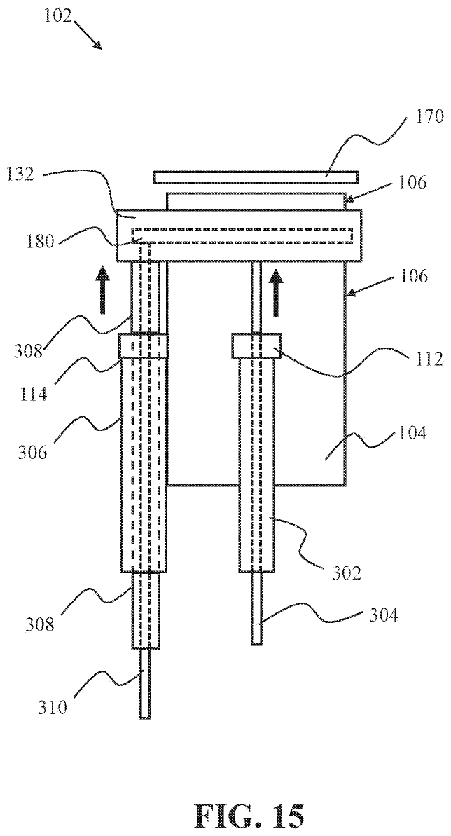

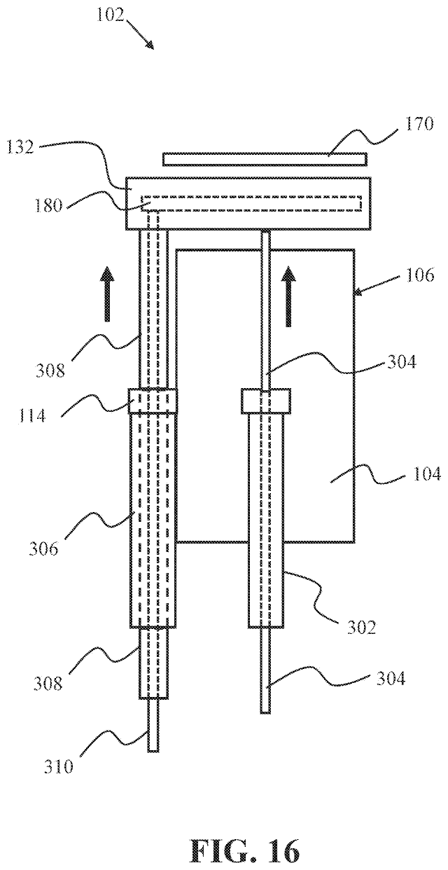

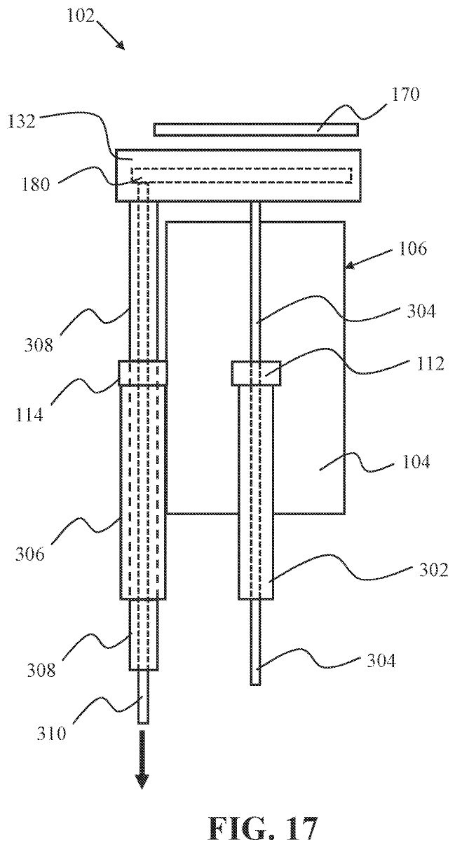

FIGS. 14-17 show schematic illustrations of the closure and retrieval assembly 102 in various stages of deployment. The closure and retrieval assembly 102 is shown in the first or undeployed configuration in FIG. 14, in the second or partially deployed configuration in FIG. 15, in the third or fully deployed configuration FIG. 16, and in the fourth or actuated configuration in FIG. 17. The closure and retrieval assembly 102 may be coupled with the catheter sheath assembly 300 to deploy and actuate the closure and retrieval assembly 102.

As shown in FIG. 14, when the closure and retrieval assembly 102 is in the undeployed configuration, the collar 132 is disposed around the outside surface of a medial portion of the housing 104. The cinch element 170 disposed around the outside surface of the housing 104 between the collar 132 and the distal end of the housing 104. The cutting element 180 is disposed in the collar 132 in an undeployed position.

One or more clip deployment catheters 302 of the catheter sheath assembly 300 may be connected to a medial portion of the housing 104 and extend proximally, such as to the actuation assembly 200. A distal end of each clip deployment catheter 302 may be coupled to a clip deployment catheter receiver 112 extending radially outwardly from the outer surface 106 of the housing 104. Each clip deployment catheter receiver 112 may include a substantially circular receiving portion configured to receive the distal end of one of the clip deployment catheters 302. Each clip deployment catheter receiver 112 may be configured to secure the distal end of the clip deployment catheter 302 to a medial portion of the housing 104 such that the distal end of the clip deployment catheter 302 and the medial portion of the housing 104 move in concert. The distal end of each clip deployment catheter 302 may be secured in one of the clip deployment catheter receivers 112 by adhesives, welding, fasteners, over-molding, or the like. In some embodiments, the housing 104 is shaped and configured to include the clip deployment catheter receiver 112. In other embodiments, the clip deployment catheter receivers 112 are separate from the housing 104 and are secured to the outer surface of the housing 104 via adhesives, welding, fasteners, over-molding, or the like.

One or more housing catheters 306 of the catheter sheath assembly 300 may also be connected to a medial portion of the housing 104 extend proximally from, such as to the actuation assembly 200. A distal end of each housing catheter 306 may be coupled to a catheter receiver 114 extending radially outwardly from the housing 104. Each catheter receiver 114 may include a substantially circular receiving portion configured to receive the distal end of one of the housing catheters 306 and may be configured to secure the distal end of the housing catheter 306 to a medial portion of the housing 104 such that the distal end of the housing catheter 306 and the medial portion of the housing 104 move in concert. The distal end of each catheter receiver 114 may be substantially in line with the distal end of each clip deployment catheter receiver 112. The distal end of each housing catheter 306 may be secured in one of the catheter receivers 114 by adhesives, welding, fasteners, over-molding, or the like. In some embodiments, the housing 104 is shaped and configured to include the catheter receivers 114. In other embodiments, the catheter receivers 114 are separate from the housing 104 and are secured to the outer surface of the housing 104 via adhesives, welding, fasteners, over-molding, or the like.

In the undeployed position, the collar 132 may be disposed on the housing 104 such that the proximal end of the collar 132 abuts the distal ends of the one or more clip deployment catheter receivers 112 and/or the distal ends of the one or more catheter receivers 114. The abutment between the collar 132 and the one or more clip deployment catheter receivers 112 and/or the one or more catheter receivers 114 may prevent proximal movement of the collar 132 relative to the housing 104. The cutting element 180 may be disposed within a radially inner portion of the collar 132, such as coupled to an inner wall of the collar 132 and/or within a receiving area in the collar 132. The clip deployment catheter receivers 112 and/or the catheter receivers 114 may be sized, shaped, and configured such that that collar 132 is prevented from sliding proximally over housing 104. For example, the clip deployment catheter receivers 112 and/or the catheter receivers 114 may extend radially outwardly from the outer surface 106 of the housing 104 to a height greater than the inner surface 134 of the collar 132.

A deployment actuation wire 304 of the catheter sheath assembly 300 may extend through each of the clip deployment catheters 302 and a distal end of the deployment actuation wire 304 may be coupled with the collar 132. The deployment actuation wires 304 may be configured to transfer translational or longitudinal movement from the actuation assembly 200 to the closure and retrieval assembly 102. The distal end of the deployment actuation wire 304 may be coupled to the collar 132 via adhesives, welding, fastenings, loops, interlocking features, or the like. The deployment actuation wire 304 may extend proximally from the collar 132, such as to the actuation assembly 200. Each deployment actuation wire 304 may move longitudinally and rotationally separately from the clip deployment catheter 302 through which the deployment actuation wire 304 is disposed. For example, when the clip deployment catheter 302 is proximally retracted, thereby moving the housing 104 proximally, the deployment actuation wire 304 may remain coupled to the collar 132 and the collar 132 may remain stationary.

A collar catheter 308 of the catheter sheath assembly 300 may extend through each of the housing catheters 306 and a distal end of each collar catheter 308 may couple with the collar 132. The collar catheter 308 may extend proximally from the collar 132, such as to the actuation assembly 200. The distal end of the collar catheter 308 may extend distally from the housing catheter 306 and may be coupled to the collar 132 via adhesives, welding, fastenings, loops, interlocking features, or the like. Each collar catheter 308 may move longitudinally and rotationally separately from the housing catheter 306 through which the collar catheter 308 is disposed. For example, when the housing catheter 306 is proximally retracted, thereby moving the housing 104 proximally, the collar catheter 308 may remain coupled to the collar 132 and the collar 132 may remain stationary.

A cutting actuation wire 310 of the catheter sheath assembly 300 may extend through each of the collar catheters 308 and couple with the cutting element 180. The cutting actuation wire 310 may be configured to transfer translational or longitudinal movement of the actuation assembly 200, such as proximal translation, to the cutting element 180. The cutting element 180 and cutting actuation wire 310 may be coupled and configured such that the cutting element 180 is actuated via actuation of the cutting actuation wire 310, such as when the cutting actuation wire 310 is pulled proximally. The cutting actuation wire 310 may extend proximally from the cutting element 180, such as with the proximal end of the cutting actuation wire 310 being coupled to the actuation assembly 200. The distal end of the cutting actuation wire 310 may extend distally from the housing catheter 306 and collar catheter 308 through which the cutting actuation wire 310 is disposed. The cutting actuation wire 310 may move longitudinally and rotate separately from the collar catheter 308 through which the cutting actuation wire 310 is disposed. For example, the cutting actuation wire 310 may be pulled proximally, thereby actuating the cutting element 180, without movement of the collar 132 or the housing 104.

The clip deployment catheters 302 may comprise high density polyethylene (HDPE), polytetrafluoroethylene (PTFE), polyetheretherketone (PEEK), fluorinated ethylene propylene (FEP), shape memory metal, such as Nitinol, stainless steel, or the like, or combinations thereof. The housing catheter 306 may comprise HDPE, PTFE, PEEK, FEP, shape memory metal, such as Nitinol, stainless steel, or the like, or combinations thereof. The collar catheter 308 may comprise HDPE, PTFE, PEEK, FEP, shape memory metal, such as Nitinol, stainless steel, or the like, or combinations thereof. The deployment actuation wires 304 and the cutting actuation wire 310 may each be a solid cable, a hollow tube, or other suitable elongated object or combination of objects, such as a drive cable, a torque cable, a hypotube, spring sheath, or a catheter. The deployment actuation wires 304 and the cutting actuation wire 310 may comprise stainless steel, shape memory metal, such as Nitinol, or combinations thereof, and may include a PTFE, HDPE, PEEK, FEP, or similar liner or coating. In a preferred embodiment, the clip deployment catheters 302 and the collar catheter 308 comprise HDPE, the housing catheter 306 comprises PTFE, the deployment actuation wires 304 comprise stainless steel and include a PTFE coating, and the cutting actuation wire 310 comprises stainless steel.

The cinch element 170 may be disposed on a distal portion of the housing 104 in the open position with the tines 174 projecting distally from the perimeter 172. The cinch element 170 may be disposed around the housing 104 such that the outer surface 106 of the housing 104 biases the tines 174 of the cinch element 170 into the open position while the cinch element 170 is disposed around the housing 104. The cinch element 170 may be disposed on the housing such that the proximal portion of the cinch element 170 abuts the distal end of the collar 132. The abutment of the collar 132 with the housing 104, such as with the distal ends of the one or more clip deployment catheter receivers 112 and/or the distal ends of the one or more catheter receivers 114, may prevent the collar 132 from moving proximally. The abutment of the cinch element 170 with the collar 132 may prevent the cinch element from moving proximally.

When the closure and retrieval assembly 102 is in the undeployed configuration at the distal end of the endoscope positioned within a human body, tissue may be recruited into the tissue receiving portion 110 of the housing 104. The recruited tissue may extend into the housing 104 past the distal end of the of the housing 104 and toward the proximal end of the housing 104. The tissue may be recruited into the tissue receiving portion 110 to a distance such that the recruited tissue may be cinched by the cinch element 170 when it is deployed, such as described below. The tissue may be recruited into the tissue receiving portion 110 such that the tissue forms a pseudo-polyp.

As shown in FIG. 15, the closure and retrieval assembly 102 may be moved from the first or undeployed position to the second or partially deployed position. When the closure and retrieval assembly 102 is in the partially deployed configuration, the collar 132 is disposed near the distal end of the housing 104. The collar 132 may be moved distally relative to the housing 104 from the undeployed configuration by distal extension or actuation of the deployment actuation wires 304 and/or the collar catheters 308. For example, proximal ends of the one or more deployment actuation wires 304 and/or the one or more collar catheters 308 may be pushed or otherwise moved distally with the housing 104 in a substantially stationary position such that the collar 132 moves distally relative to the housing 104. The proximal ends of the one or more housing catheters 306 and/or the one or more clip deployment catheters 302 may be fixed, such as to the actuation assembly 200, such that the housing 104 remains substantially stationary when the collar 132 is moved distally. The one or more collar catheter 308 and the one or more deployment actuation wires 304 may extend distally out of the housing catheter 306 and the clip deployment catheter 302, respectively, to move the collar 132 distally relative to the housing 104.

The movement of the collar 132 may move the cinch element 170 distally relative to the housing 104 such that the cinch element 170 is moved beyond the distal end of the housing 104. The cinch element 170 may move from the undeployed position to the deployed position as the cinch element 170 is moved distally off the housing 104. When the cinch element 170 is moved beyond the distal end of the housing 104, the housing 104 no longer biases the tines 174 of the cinch element 170 radially outwardly and the tines 174 may move radially inwardly to the closed position. The tines 174 may pivot radially inwardly about the perimeter 172 such that the tines 174 come substantially together at the center of the cinch element 170, such as to secure tissue recruited into the tissue receiving portion between the tines 174. The cinch element 170 may cinch the tissue recruited into the tissue receiving portion 110 of the housing 104 when the cinch element 170 is deployed from the housing 104 and moved to the closed position. Additionally, the tissue recruitment mechanism, such as grasping devices and/or suction, may continue to recruit the tissue in the tissue receiving portion 110 after the cinch element 170 is deployed from the housing 104.

As shown in FIG. 16, the closure and retrieval assembly 102 may be moved from the partially deployed to the fully deployed configuration. When the closure and retrieval assembly 102 is in the fully deployed configuration, the collar 132 is disposed distally past the distal end of the housing 104. The collar 132 may be moved distally relative to the housing 104 from the partially deployed configuration by farther distal extension or actuation of the one or more deployment actuation wires 304 and/or the one or more collar catheters 308. For example, the distal ends of the one or more deployment actuation wires 304 and/or the one or more collar catheters 30 may be pushed or otherwise moved farther distally with the housing 104 in a substantially stationary position such that the collar 132 moves distally past to the housing 104. The one or more collar catheters 308 and the one or more deployment actuation wires 304 may extend father distally out of the housing catheter 306 and the clip deployment catheter 302, respectively, to move the collar 132 distally relative to the housing 104.

The inner surface 134 of the collar 132 may be sized, shaped, or otherwise configured such that the cinch element 170 is deployed from the housing 104 before the collar 132 is moved distally beyond the distal end of the housing 104. The inner diameter of the inner surface 134 of the collar 132 may be less than the outer diameter of the cinch element 170 such that distal movement of the collar 132 pushes the cinch element 170 off the housing 104 before the collar 132 may be extended beyond the distal end of the housing 104. For example, the cinch element 170 and collar 132 may be configured such that the cinch element 170 must be deployed from the housing 104 before the collar 132 may move distally beyond the distal end of the housing 104.

In the fully deployed configuration, the collar 132 may be disposed proximally to the deployed cinch element 170, such as above the cinched tissue. The collar 132 may have an inner diameter smaller than the outer diameter of the perimeter 172 of the cinch element 170 such that the collar 132 is not moved distally beyond the deployed cinch element 170. The collar 132 and/or cinch element 170 may be sized, shaped, and configured such that the cutting element 180 is disposed in the desired position in relation to the deployed cinch element 170, the housing 104, and/or the target tissue when the closure and retrieval assembly 102 is in the fully deployed configuration.

While the collar 132 has been described as being pushed distally relative to a stationary housing 104, it will be understood that the collar 132 may move the cinch element 170 in other manners. For example, the collar 132 may be moved distally relative to the housing 104 by proximal retraction of the housing 104, such as via proximal retraction of the one or more clip deployment catheters 302 and the one or more housing catheters 306.

As shown in FIG. 17, when the closure and retrieval assembly 102 is in the fully deployed configuration, the cutting element 180 may be actuated, such as to cut the tissue recruited into the tissue receiving portion 110 of the housing 104 proximally from the deployed cinch element 170. In the illustrated embodiment, the cutting element 180 is actuated by proximal movement of the cutting actuation wire 310. In other embodiments, the cutting element 180 is actuated by additional sutures, actuation lines, or tethers, by mechanical actuation, or via energization. The actuation of the cutting element 180 may cut tissue proximally from the deployed cinch element 170 such that uncut tissue remains cinched by the cinch element 170. The tissue recruited into the housing 104 may continue to be recruited toward the housing, such as via suction, forceps, or the like, such that the severed tissue may remain within the tissue receiving portion 110.

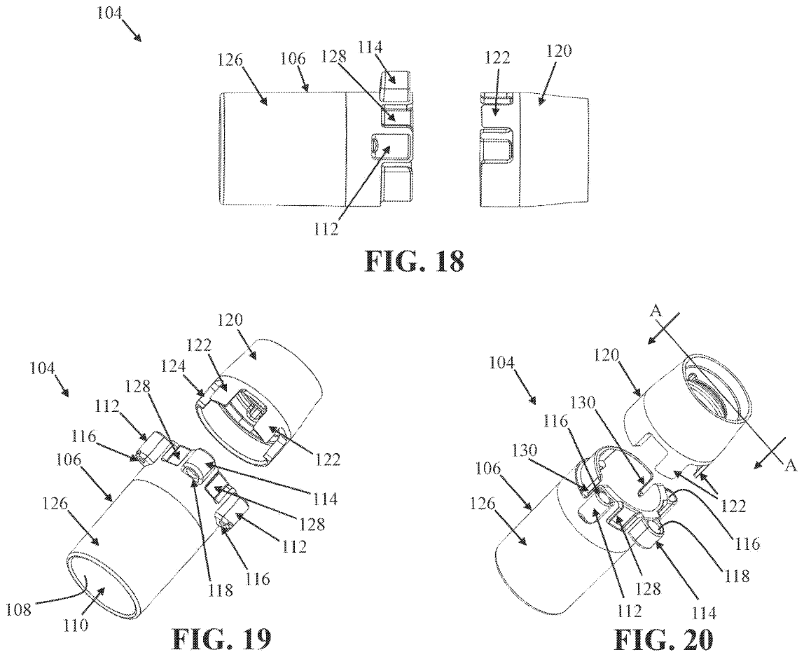

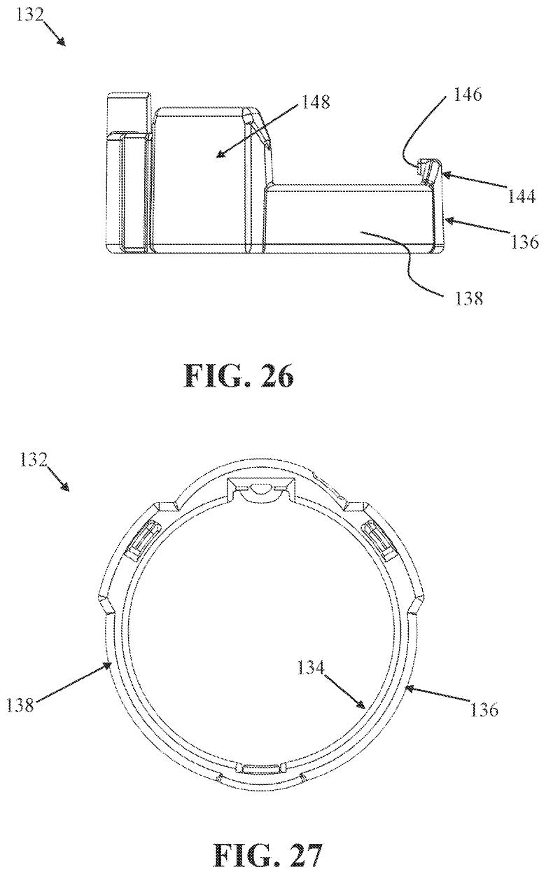

Referring now to FIGS. 18-33, a closure and retrieval assembly 102 is shown according to one embodiment. As shown in FIGS. 18-23, the housing 104 includes a receiving portion 126 and a coupling portion 120 configured to couple with a proximal end of the receiving portion 126. The receiving portion 126 is a substantially hollow cylinder and an inner surface of the receiving portion 126 defines the tissue receiving portion 110. The inner surface of the receiving portion 126 may be larger than the distal end of an endoscope. In some embodiments, the outer surface of the receiving portion 126 is tapered distally. The distal taper of the receiving portion 126 may assist in the deployment of the cinch element 170 and the collar 132 during operation of the closure and retrieval assembly 102.

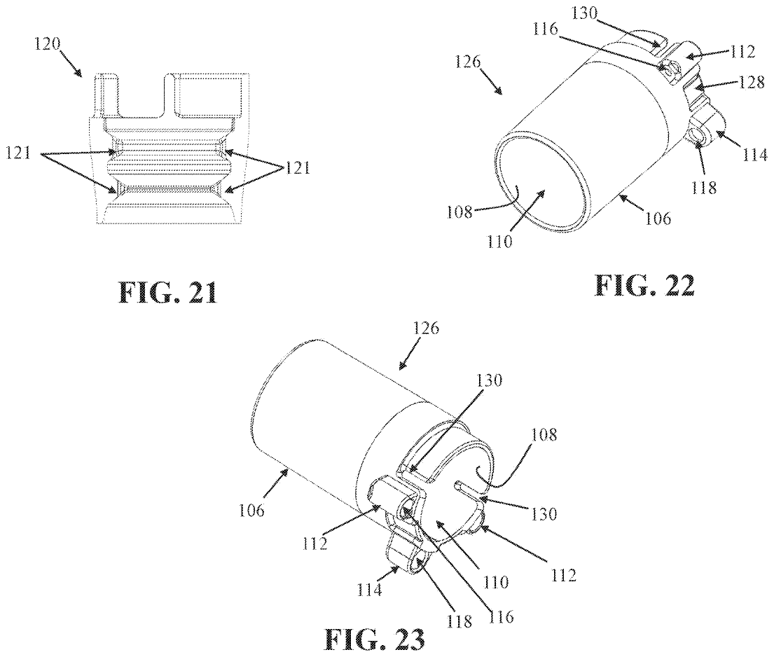

The coupling portion 120 is tapered proximally and is configured to couple the housing 104 to the distal end of an endoscope such that the coupling portion 120 is disposed around the distal end of the endoscope. In some embodiments, the coupling portion 120 may be press fit to the distal end of the endoscope. Additionally or alternatively, the coupling portion 120 may be coupled to the distal end of the endoscope with adhesives, welding, over-molding, or the like. As shown in FIG. 21, the inner surface of the coupling portion 120 may define or include one or more ribs 121 extending radially inwardly and configured to secure the coupling portion 120 to the distal end of the endoscope. The ribs 121 may be sized, shaped, and configured to secure coupling portion 120 to the distal end of the endoscope when the distal end of the endoscope is disposed in the coupling portion 120.

In some embodiments, the receiving portion 126 comprises a hard plastic. Additionally or alternatively, the receiving portion 126 may comprise acrylonitrile butadiene styrene (ABS), styrene-butadiene copolymer (SBC), polycarbonate, or acrylic, or any combinations thereof. The receiving portion 126 may be at least partially transparent or clear such that the contents of the tissue receiving portion 110 may be viewed from outside the receiving portion 126 and/or such that an operator may observe the portions of the body outside of the receiving portion 126.

In some embodiments, the coupling portion 120 comprises a thermoplastic elastomer (TPE), such as a soft TPE, and/or thermoplastic polyurethane (TPU). However, it will be understood that the receiving portion 126 and the coupling portion 120 may comprises one or more other suitable materials. For example, the receiving portion 126 and/or the coupling portion 120 may comprise stainless steel, HDPE, low density polyethylene (LDPE), ultra high molecular weight polyethylene (UHMW), PTFE, polyvinyl chloride (PVC), rubber, such as liquid silicone rubber, or the like, or any combinations thereof.

The coupling portion 120 and the receiving portion 126 may be coupled together to form the housing 104. In some embodiments, the coupling portion 120 is over-molded to the proximal end of the receiving portion 126. Additionally or alternatively, the coupling portion 120 may be coupled to the receiving portion 126 via press-fitting, snap-fitting, fasteners, welding, adhesives, or combinations thereof.

In the illustrated embodiment, the coupling portion 120 includes one or more teeth 122 extending distally from the remainder of the coupling portion 120 and one or more posts 124 extending radially inwardly from the remainder of the inner surface. The receiving portion 126 includes one or more depressions 128 in the outer surface near the proximal end of the receiving portion 126 and one or more slots 130 extending distally into the proximal end of the receiving portion 126. When the coupling portion 120 is coupled with the receiving portion 126, the teeth 122 of the coupling portion 120 are disposed in the depressions 128 of the receiving portion 126 and the posts 124 of the coupling portion 120 are received in the slots 130 of the receiving portion 126. The coupling of the teeth 122 and the depressions 128 may secure the coupling portion 120 with the receiving portion 126. The coupling of the posts 124 and the slots 130 may prevent the coupling and receiving portions 120, 126 from rotating relative to each other. The slots 130 may also permit the proximal end of the receiving portion 126 to couple with or otherwise accommodate endoscopes of various sizes.

The receiving portion 126 includes one or more clip deployment catheter receivers 112 extending radially outwardly from the outer surface 106. Each clip deployment catheter receiver 112 may include a wire passage 116 extending therethrough. The wire passage 116 may have a substantially circular inlet at the proximal end and a substantially circular outlet at the distal end. The inlet of the wire passage 116 may be larger than the outlet. The inlet of the wire passage 116 may be configured to receive one of the clip deployment catheters 302. The outlet of the wire passage 116 may be configured to permit one of the deployment actuation wires 304 to pass therefrom. The outlet of the wire passage 116 may be sized, shaped, or configured to prevent the clip deployment catheter 302 inserted into the opening from exiting the wire passage 116 through the outlet. One of the clip deployment catheters 302 with a deployment actuation wire 304 extending therethrough may be inserted into the inlet of the wire passage 116 and the deployment actuation wire 304 may extend distally from the outlet of the wire passage 116 with the distal end of the clip deployment catheter 302 prevented from exiting the wire passage 116. The distal end of the clip deployment catheter 302 may be secured in the wire passage 116 with adhesives, welding, fasteners, press-fitting, over-molding or the like.

The receiving portion 126 also includes one or more catheter receivers 114 extending radially outwardly from the outer surface 106. Each catheter receiver 114 includes a catheter passage 118 extending therethrough and having a substantially circular inlet at the proximal end and substantially circular outlet at the distal end. The inlet of the catheter passage 118 may be larger than the outlet. The inlet of the catheter passage 118 be configured to receive one of the housing catheters 306. The outlet of the catheter passage 118 be configured to permit a collar catheter 308, such as a collar catheter 308 with a cutting actuation wire 310 extending therethrough, to pass therefrom. The outlet of the catheter passage 118 may also be sized, shaped, or configured to prevent the housing catheter 306 inserted into the opening from exiting the catheter passage 118 through the outlet. One of the housing catheters 306 with a collar catheter 308 extending therethrough may be inserted into the inlet of the catheter passage 118 and the collar catheter 308 may extend distally from the outlet of the catheter passage 118 with the distal end of the housing catheter 306 prevented from exiting the catheter passage 118. The distal end of the housing catheter 306 may be secured in the catheter passage 118 with adhesives, welding, fasteners, press-fitting, over-molding or the like. In the illustrated embodiment, the housing 104 includes one catheter receiver 114.

The clip deployment catheter receivers 112 and the catheter receivers 114 may be configured and disposed around the outer surface 106 of the housing 104 to increase operational control of the closure and retrieval assembly 102 and/or to decrease operational forces acting upon the closure and retrieval assembly 102 during deployment. In the illustrated embodiment, the housing 104 includes one catheter receiver 114 and two clip deployment catheter receivers 112. The clip deployment catheter receivers 112 are disposed substantially equidistantly on opposite sides of the catheter receiver 114. In one embodiment, each clip deployment catheter receiver 112 is disposed about 55° along the circumference of the outer surface 106 of the housing 104 from the catheter receiver 114. However, it will be understood that the closure and retrieval device 100 may include any suitable number of clip deployment catheter receivers 112 and catheter receivers 114, and the clip deployment catheter receivers 112 and catheter receivers 114 may be disposed in any suitable configuration. For example, the housing 104 may include a number of catheter receivers 114 and a number of clip deployment catheter receivers 112 corresponding to the number of clip deployment catheters 302 and housing catheters 306, respectively, included in the catheter sheath assembly 300. Additionally, while the receiving portion 126 of the housing 104 has been described as being separate from the coupling portion 120, it will be understood that the coupling and receiving portions 120, 126 may be integral.

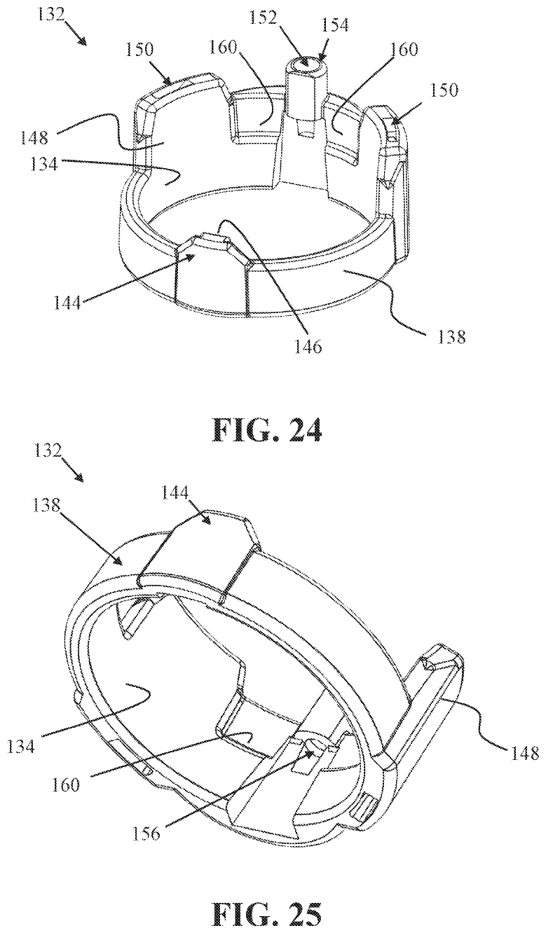

As shown in FIGS. 24-27, the collar 132 includes a ring portion 138 at a distal end and a connecting portion 148 extending proximally from the ring portion 138. The ring portion 138 may be configured to be slidably disposed around the outside of the housing 104. The ring portion 138 may be substantially circular and may define the inner surface 134 and the outer surface 136 of the collar 132. The inner surface 134 of the ring portion 138 may have a diameter larger than a diameter of the outer surface 106 of the housing 104. The inner surface 134 of the ring portion 138 may also have a diameter less than a diameter of the outer surface 106 of the housing 104 combined with either the catheter receivers 114 or the clip deployment catheter receivers 112. The ring portion 138 may have a height in the longitudinal direction (e.g., from the distal end to the proximal end) substantially corresponding to the distance from the deployed cinch element 170 that the target tissue is to be cut when the closure and retrieval assembly 102 is actuated. In some embodiments, the ring portion 138 has a height between about 1 mm and about 6 mm, such as between about 2 mm and about 5 mm, such as about 4 mm.

The connecting portion 148 may be configured to couple with the distal ends of the deployment actuation wires 304 and the collar catheter 308. The connecting portion 148 may be substantially arcuate such that the radially inner surface of the connecting portion 148 does not extend radially inwardly past the inner surface 134 of the ring portion 138. The connecting portion 148 may include one or more pockets 150 configured to receive and couple with the distal end of one of the deployment actuation wires 304. Each pocket 150 may extend distally into the connecting portion 148 from the proximal end of the connecting portion 148. The pockets 150 may be sized, shaped, and configured to receive and couple with the distal end of the deployment actuation wires 304. The connecting portion 148 may include a number of pockets 150 positioned around the connecting portion 148 corresponding to the number of clip deployment catheter receivers 112 disposed around the housing 104 such that each deployment actuation wire 304 may extend substantially straight from the outlet of one of the clip deployment catheter receivers 112 to one of the pockets 150.

The connecting portion 148 may include one or more actuation passages 152 extending from a proximal opening 154 to a distal opening 156 near the ring portion 138. The actuation passage 152 may be configured to receive and couple with the distal end of the collar catheter 308 and to permit the cutting actuation wire 310 to pass therethrough and/or to permit a portion of the cutting element 180 to extend proximally into the actuation passage 152. The proximal opening 154 may be larger than the distal opening 156. The proximal opening 154 may be sized, shaped, and configured to receive the distal end of the collar catheter 308. The distal opening 156 may be sized, shaped, and configured to permit the distal end of the cutting actuation wire 310 to exit from the actuation passage 152 and/or to permit a proximal portion of the cutting element 180 to extend proximally into the actuation passage 152. The distal opening 156 may also be sized, shaped, and configured to prevent the distal end of the collar catheter 308 from passing distally through the actuation passage 152. For example, the distal opening of the actuation passage 152 may have a diameter greater than both the diameter of the cutting actuation wire 310 and the diameter of a portion of the cutting element 180 and less than the diameter of the collar catheter 308.

The connecting portion 148 may include a number of actuation passages 152 positioned around the connecting portion 148 corresponding to the number of catheter receivers 114 disposed around the housing 104 such that each collar catheter 308 and cutting actuation wire 310 may extend substantially straight from the outlet of the catheter receiver 114 into the actuation passage 152.

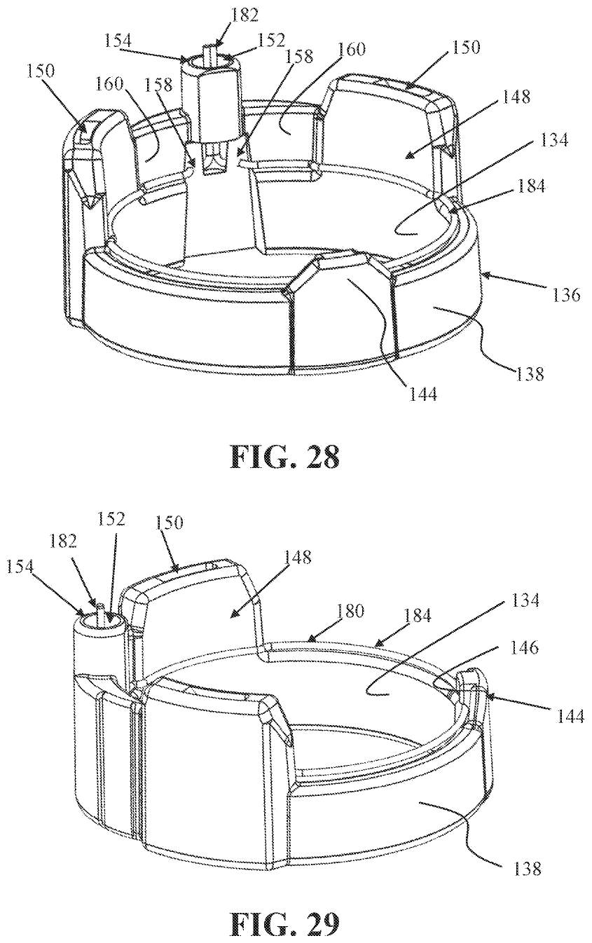

In some embodiments, the connecting portion 148 also includes one or more internal passages 158 (FIG. 28) extending through a portion of the connecting portion 148. The internal passages 158 may have an opening disposed near the distal opening 156 of the actuation passage 152. The internal passages 158 may be configured to retain a portion of the cutting element 180 or the cutting actuation wire 310 therethrough.

In some embodiments, the connecting portion 148 includes one or more recesses 160 extending radially into the inner surface of the connecting portion 148 on one or both sides of the actuation passage 152. The recesses 160 may extend radially into the inner surface of the connecting portion 148 beyond the inner surface 134 of the ring portion 138. The recesses 160 may be positioned and configured such that a portion of cutting element 180 may be disposed within at least one of the recesses 160 and above a portion of the ring portion 138 such that the cutting element 180 is prevented or otherwise restricted from moving distally by abutment with the proximal end of the ring portion 138.

In some embodiments, the ring portion 138 includes an extension 144 extending proximally from the remainder of the ring portion 138. The extension 144 may be disposed substantially opposite the connecting portion 148. The extension 144 may include a ledge 146 extending radially inwardly from a proximal end of the extension 144. The ledge 146 may be configured to at least partially retain the cutting element 180 in position in relation to the collar 132. For example, a portion of the cutting element 180 may be disposed distally from the ledge 146 and proximally from remainder of the ring portion 138 such that the cutting element 180 is maintained in position between the ledge 146 and the remainder of the ring portion 138.

As shown in FIGS. 28-31, the cutting element 180 may be disposed within the collar 132, such as when the closure and retrieval assembly 102 is in the unactuated position. In the illustrated embodiment, the cutting element 180 is a loop or snare which may constrict to cut tissue. However, it will be understood that the cutting element 180 of the closure and retrieval device 100 of the present disclosure may include other cutting instruments or forms of severing tissue, such as described below.

The cutting element 180 may be disposed within the collar 132 such that the cutting element 180 may be actuated to cut or otherwise sever tissue after the collar 132 has been moved distally off of the housing 104. The cutting element 180 may include a loop 184 and one or more legs 182. The loop 184 may be actuated by proximal movement of the legs 182. For example, the legs 182 may be coupled to the distal end of the cutting actuation wire 310 such that proximal movement of the cutting actuation wire 310 actuates the cutting element 180. The cutting element 180 may be a wire, tether, cable, or the like which is configured to constrict or otherwise cut tissue. In a preferred embodiment, the cutting element 180 is a stainless steel cable.

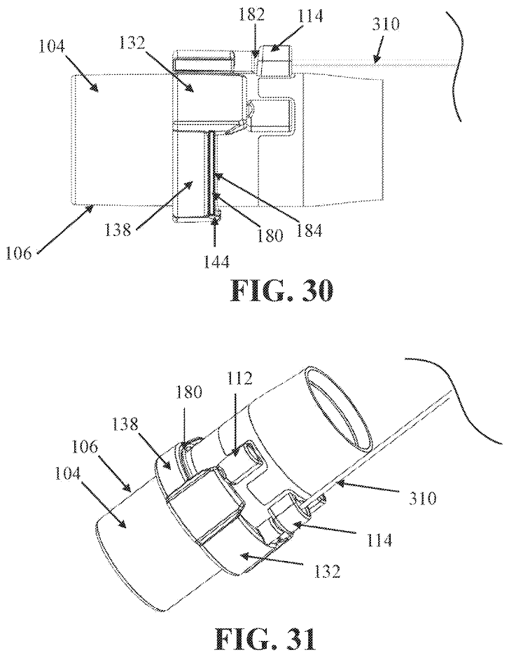

The legs 182 of the cutting element 180 may extend into or through the actuation passage 152 of the connecting portion 148. The loop 184 may be disposed within the collar 132 such that the loop 184 may be disposed around the housing 104 when the collar 132 is disposed around the housing 104 (FIGS. 30-31). For example, the loop 184 may be disposed substantially proximally to the ring portion 138 and may extend around the inner surface 134 of the collar 132. The loop 184 may be disposed substantially 90° relatively to a longitudinal axis extending through the legs 182.

In some embodiments, part of the loop 184 is disposed distally to the ledge 146 of the extension 144 of the collar 132. The loop 184 may be disposed between the ledge 146 of the extension 144 and the remainder of the ring portion 138. The loop 184 may be held in place between the ledge 146 and the remainder of the ring portion 138 during deployment and until the cutting element 180 is actuated. For example, the loop 184 may remain between the ledge 146 and the remainder of the ring portion 138 during deployment and the loop 184 may be pulled radially inwardly and out from between the ledge 146 when the cutting element 180 is actuated.

In some embodiments, part of the loop 184 is bent or otherwise configured such that part of the loop 184 is disposed in one or more recesses 160 in the collar 132. The bent portions of the loop 184 may be disposed proximally to the ring portion 138. The positioning of the loop 184 within the recesses 160 may prevent or otherwise restrict the cutting element 180 from moving distally past the collar 132.

In some embodiments, part of the loop 184 extend through one or more internal passages 158 of the collar 132. The positioning of the loop 184 within the internal passages 158 may prevent the cutting element 180 from decoupling with the collar 132. The loop 184 may extend through the internal passages 158 near the actuation passage 152, such as opposite the ledge 146, such that the loop 184 remains substantially perpendicular to a longitudinal axis extending through the collar 132. The extension of the loop 184 through the internal passages 158 may also prevent the cutting element 180 from being pulled proximally out of the collar 132.

Referring now to FIGS. 30-33, the collar 132 and the cutting element 180 may disposed around the housing 104 such that the collar 132 may be moved from the undeployed to deployed positions and the cutting element 180 may be actuated. As shown in FIGS. 30-31, in the unactuated configuration, the collar 132 may be disposed around the outer surface 106 of the housing 104 distally from the catheter receiver 114 and the clip deployment catheter receivers 112. The cutting element 180 may be disposed partially within the collar 132. The loop 184 of the cutting element 180 may extend substantially around the outer surface 106 of the housing 104. A portion of the loop 184 may be disposed between the ledge 146 and the remainder of the ring portion 138. The proximal end of the cutting element 180 may be coupled with the distal end of the cutting actuation wire 310.

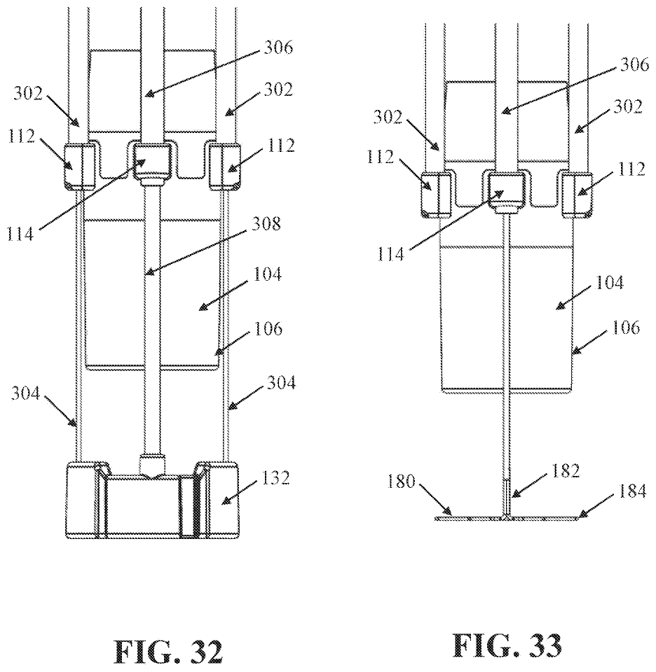

As shown in FIGS. 32-33, the collar 132 and the cutting element 180 may be moved from the undeployed configuration to the deployed configuration, with the collar 132 (as well as the deployment actuation wires 304 and the collar catheter 308) being removed in FIG. 33. The collar 132 may be moved distally relative to the housing 104 via distal extension of the deployment actuation wires 304 from the clip deployment catheters 302 and or distal extension of the collar catheter 308 from the housing catheter 306. The cutting element 180 may move distally with the collar 132 and may remain disposed in the collar 132. For example, part of the loop 184 may remain between the ledge 146 and the remainder of the ring portion 138. The loop 184 may be disposed at substantially a right angle to the legs 182 when the closure and retrieval assembly 102 is in the deployed configuration. When the cutting element 180 is actuated, the loop 184 may constrict, such as via proximal movement of the legs 182 through the actuation passage 152, which may sever target tissue, such as tissue cinched via the cinch element 170.

Referring back to FIGS. 9-13, the cinch element 170 includes a plurality of tines 174 that extend radially inwardly from the perimeter 172. The plurality of tines 174 may come together at a center portion of the cinch element 170 when the cinch element 170 is in the closed position. The radially innermost portion of each tine 174 may be sharp, such as to pierce tissue. For example, the radially innermost portion of each tine 174 may be sharp enough to pierce a wall of a colon. The tines 174 may be sized, shaped, and configured such that the tines 174 may pierce tissue without grasping anything on the other side of the pierced tissue.

The tines 174 may include a neck 176 near the perimeter 172 which is configured to bend or flex outwardly to move the cinch element 170 to the open position. The radially inner portions of the tines 174 may be jagged or leaf-shaped, such as to increase the grip of the cinch element 170 on tissue. When moved to the closed position, the tines may come together like pieces of a puzzle for better grip on tissue. The tines 174 may be sized, shaped, and configured to define one or more gaps 178 between the tines 174 when the cinch element 170 is in the closed position. The gaps 178 may be used to increase retention of tissue and to allow some circulation in the clipped tissue.

In the illustrated embodiment, the cinch element 170 is a hexagon with concave edges. However, it will be understood that the cinch element 170 may have any suitable shape or configuration. For example, the cinch element 170 may be a circle, an oval, a triangle, a square, a pentagon, or any other suitable shape and the edges may be rounded, concave, or convex.

As shown in FIG. 13, one side of the radially innermost portion of the tines 174 may be tapered, such as to permit smooth intubation onto tissue. Additionally, the cinch element 170 may include an inner chamfer or radius on the flat side of the cinch element 170 that may improve deployment forces.

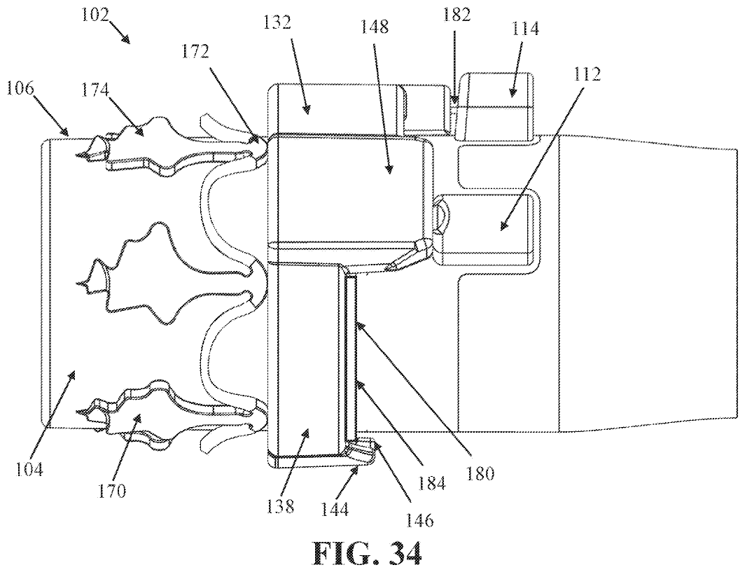

Referring now to FIG. 34, the closure and retrieval assembly 102 may be positioned in the unactuated configuration, such as when the closure and retrieval assembly 102 is inserted into the body and placed near the target tissue. The collar 132, the cutting element 180, and the cinch element 170 are disposed around the outer surface 106 of the housing 104 distally from the clip deployment catheter receivers 112 and the catheter receiver 114. The proximal end of the connecting portion 148 of the collar 132 may abut the distal end of the clip deployment catheter receivers 112. The inner diameter of the collar 132 is less than the outer diameter of the clip deployment catheters 302 such that abutment between the collar 132 and clip deployment catheter receivers 112 prevents the collar 132 from moving proximally beyond the clip deployment catheter receivers 112. The cinch element 170 is disposed in the open position distally to the collar 132. The diameter of the cinch element 170 in the open position is less than the outer diameter of the collar 132 such that abutment between the proximal end of the cinch element 170 and the distal end of the ring portion 138 prevents the cinch element 170 from moving proximally beyond the collar 132. The cutting element 180 is disposed distally to the ledge 146 of the collar 132 and proximally to the remainder of the ring portion 138 of the collar 132.

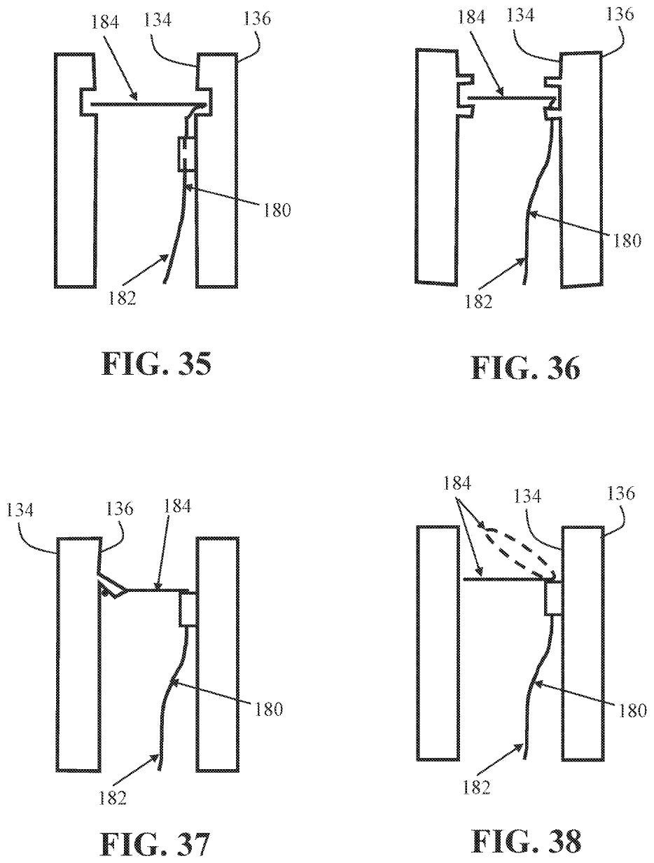

FIGS. 35-46D depict various embodiments of the cutting element 180 and the collar 132. Any combination of these embodiments, as well as any combination of with any other cutting elements 180 and collars 132 described herein, such as the cutting element 180 and collar 132 of FIGS. 28-33, can be made unless specifically excluded. Additionally, any combination of cutting element 180 and collar 132 may be combined with any suitable deployment element, such as any catheter, and any actuation element, such as the cutting actuation wires 310 described herein.

FIGS. 35-45B depict embodiments in which the cutting element 180 is a snare with one or more legs 182 and a loop 184. The cutting element 180 may be actuated by proximal movement of the legs 182, such as via the cutting actuation wire 310, which causes the loop 184 to tighten. As shown in FIG. 35, the collar 132 may include a passage or tube disposed on the inner surface 108 and a recessed channel or groove disposed circumferentially within the inner surface 134 of the collar 132 near the distal end. The leg 182 of the cutting element 180 may extend through the passage and the loop 184 may be disposed within the recessed channel before actuation. The loop 184 may have a diameter in the unactuated position that is greater than the diameter of the inner surface 134 of the collar 132 and less than the diameter of the recessed channel. The loop 184 may be removed from the groove when the cutting element 180 is actuated.

As shown in FIG. 36, the collar 132 may include two radially inwardly extending ribs or projections extending circumferentially around the inner surface 134 of the collar 132 near the distal end. The ribs may define a raised groove or channel. The loop 184 of the cutting element 180 may be disposed within the raised channel before actuation. The loop 184 may have a diameter in the unactuated position that is greater than the diameter of the ribs and less than the diameter of the inner surface 134 of the collar 132. The loop 184 may be removed from the groove when the cutting element 180 is actuated.

As shown in FIG. 37, the collar 132 may include a passage or tube disposed on the inner surface 134 of the collar 132 and a hook disposed on the inner surface 134 substantially opposite the passage. In the illustrated embodiment, the hook extends proximally. In another embodiment, the hook extends distally. The leg 182 of the cutting element 180 may extend through the passage and a portion of the loop 184 may be disposed between the hook and the inner surface 134 of the collar 132 in the unactuated position. The hook may be configured such that the loop 184 is pulled away from engagement with the hook when the cutting element 180 is actuated.

As shown in FIG. 38, the collar 132 includes a passage or tube disposed on the inner surface 134 of the collar 132. The leg 182 of the cutting element 180 may extend through the passage and the loop 184 may be relatively unsecured within the distal end of the collar 132. In some embodiments, the size, geometry, and composition of the cutting element 180 may maintain the loop 184 in place in the distal end of the collar 132 in the unactuated position.

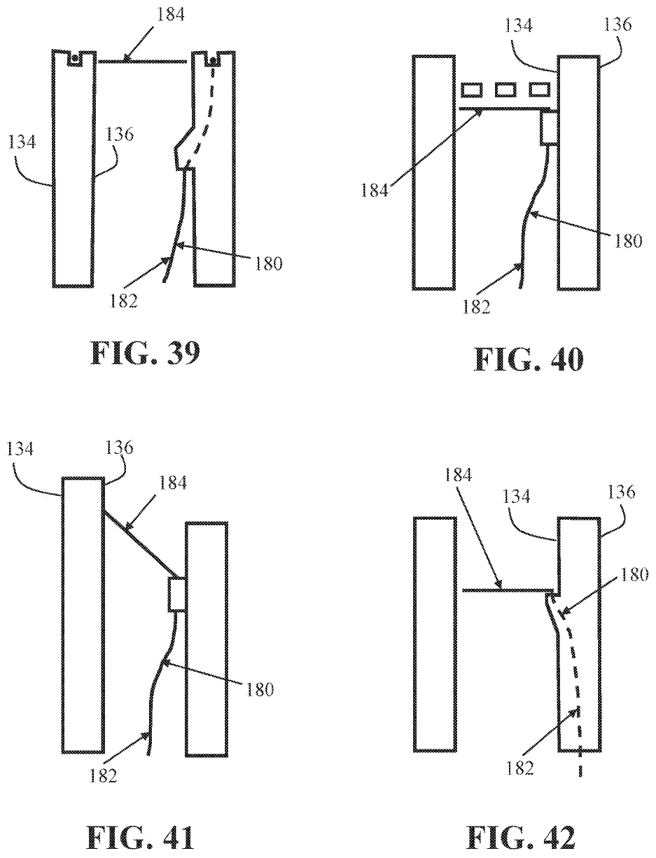

As shown in FIG. 39, the collar 132 includes a groove or channel extending into the distal end of the collar 132 and extending circumferentially around the end of the collar 132. The collar 132 may also include a channel extending from the inner surface 134 of the collar 132 to the distal groove. The leg 182 of the cutting element 180 may extend through the passage to the distal groove and the loop 184 may be disposed in the distal groove in the unactuated position. The loop 184 may have a diameter substantially equal to that of the distal groove in the unactuated position. The loop 184 may rest in the channel in the unactuated position and may be pulled out of the channel by proximal actuation of the leg 182. In some embodiments, the inner surface of the distal groove may be rounded or angled to facilitate movement of the loop 184 out of the channel when the cutting element 180 is actuated.

As shown in FIG. 40, the collar 132 includes a passage or tube and a plurality of nubs or projections extending radially inwardly and extending circumferentially around the inner surface 134 of the collar 132 near the distal end of the collar 132. The leg 182 may extend through the passage and the loop 184 may be disposed proximally to the plurality of nubs. The loop 184 may have a diameter in the unactuated position that is larger than the diameter of the nubs such that the nubs prevent the loop from moving distally past the nubs when the loop 184 is in the unactuated position. In some embodiments, the collar 132 also includes a plurality of nubs extending radially inwardly and extending circumferentially around the inner surface 134 of the collar 132 proximal to the desired position of the loop 184. While the illustrated embodiment includes a plurality of nubs, it will be understood that the collar 132 may include a plurality of hooks or other intermittent features which prevent the loop 184 from moving distally within the collar 132.

As shown in FIG. 41, one side of the collar 132 extends distally farther than the opposite side of the collar 132 such that the distal end of the collar 132 is angled. The collar 132 may also include a passage on the inner surface 134 of the collar 132, such as on the shorter side of the collar 132. The leg 182 may extend through the passage and the loop 184 may be disposed at an angle toward the longer side of the collar 132.

As shown in FIG. 42, the collar 132 includes a passage extending through one side of the collar 132 to a point on the inner surface 134 near the distal end. The leg 182 of the cutting element 180 may extend through the passage extending through the side wall of the collar 132 and the loop 184 may be disposed distally to the exit to the passage. The loop 184 may have a diameter in the unactuated position that is greater than the diameter of the inner surface 134 at the exit to the passage.

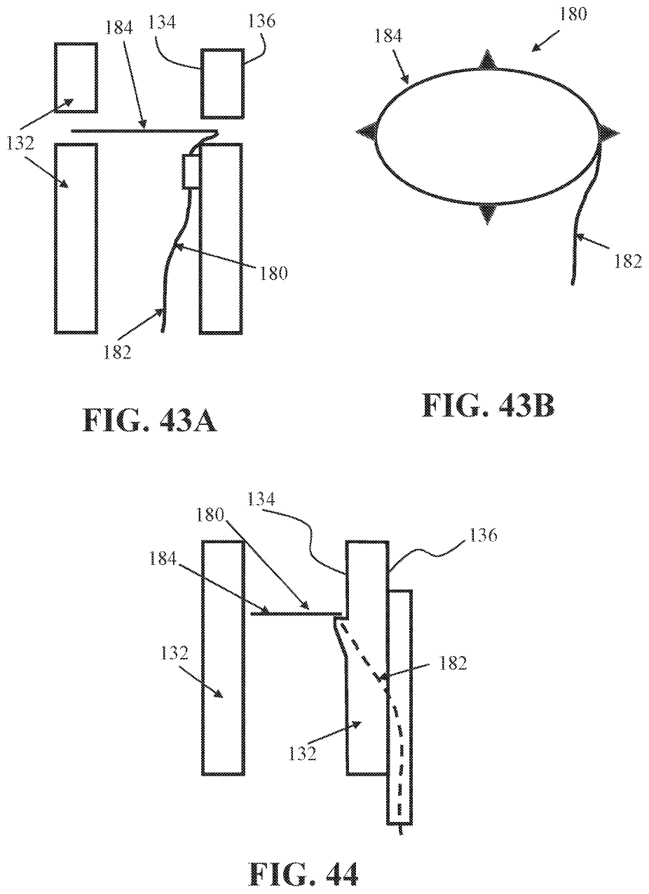

As shown in FIGS. 43A and 43B, the collar 132 may include a gap in the inner surface 134 near the distal end and a passage or tube on the inner surface 134 proximal to the gap. The leg 182 of the cutting element 180 may extend through the passage and the loop 184 may be disposed in the gap. The loop 184 may include a plurality of bends or projections extending radially outward from the remainder of the loop 184 to increase the effective diameter of the loop 184. The diameter of the loop 184 with the projections in the unactuated position may be greater than the diameter of the inner surface 134 of the collar 132 such that the loop 184 is maintained in the gap in the unactuated position. The loop 184 may be removed from the gap when the cutting element 180 is actuated.

As shown in FIG. 44, the collar 132 may include a passage extending from the outer surface 136 to the inner surface 134 of the collar 132. The leg 182 of the cutting element 180 may extend through the passage through the collar 132 and the loop 184 may be disposed distally to the exit to the passage. The leg 182 may be provided to the inlet of the passage via a catheter, a tube, a sheath, or other suitable additional component. The loop 184 may have a diameter in the unactuated position that is greater than the diameter of the inner surface 134 at the exit to the passage and less than the diameter of the inner surface 134 distal to the exit to the passage.

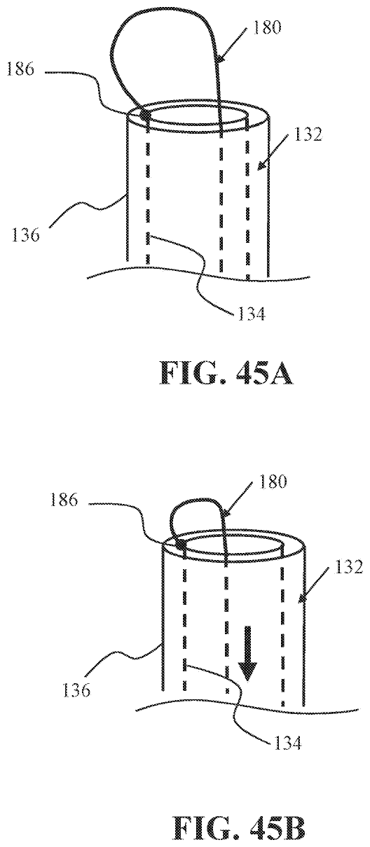

As shown in FIGS. 45A and 45B, the cutting element 180 is a snare with a distal end of the snare coupled with the distal end of the collar 132. The distal end of the cutting element 180 may be attached to the distal end of the collar 132 at a pivot 186. In other embodiments, the distal end of the cutting element 180 is coupled with the outer surface 136 of the collar 132. A proximal portion of the cutting element 180 may be coupled with the cutting actuation wire 310 (not shown). Upon actuation, the cutting element 180 may tighten against the inner surface 134 of the collar 132, such as directly opposite the pivot 186, such that the cutting element 180 may slice or otherwise sever tissue in the collar 132, such as tissue recruited into the tissue receiving portion 110.

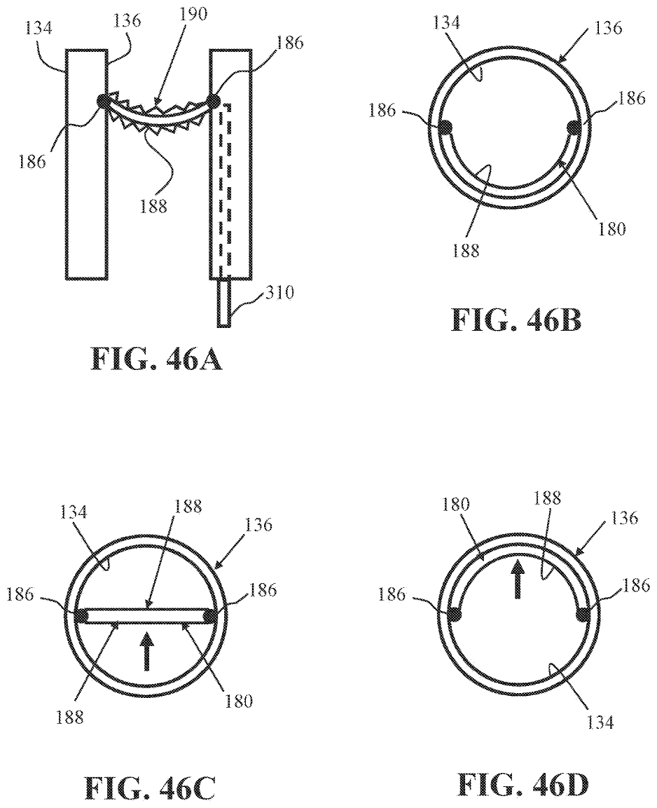

As shown in FIGS. 46A-46D, the cutting element 180 may be solid and pivotally connected to the inner surface 134 of the collar 132 such that the cutting element 180 may be rotated to cut or sever tissue. The cutting element 180 may be a blade, such as a curved blade, pivotally connected on opposite sides of the inner surface 134 of the collar 132. Each end of the cutting element 180 may be connected to the inner surface 134 of the collar 132 at a pivot 186 such that the cutting element 180 may be rotated about the pivots 186. The cutting element 180 may have two sides 188 extending between the two pivots 186. One or both of the sides 188 of the cutting element 180 may be sharpened to assist in cutting or severing tissue. In some embodiments, one or more of the sides 188 of the cutting element 180 include teeth or serrations 190 to assist in severing the target tissue when the cutting element 180 is actuated.

As shown in FIG. 46B, in the unactuated position, the cutting element 180 may extend substantially along a side of the inner surface 134 of the collar 132 such that the cutting element 180 is substantially perpendicular to a longitudinal axis extending through the collar 132. As shown in FIG. 46C, the cutting element 180 may be actuated to pivot about the two pivots 186 toward the opposite side of the inner surface 134 of the collar 132 such that the cutting element 180 extends across the medial portion of the collar 132 between the two pivots 186. In some embodiments, the cutting element 180 is rotated distally toward the distal end of the collar 132. In other embodiments, the cutting element 180 is rotated proximally toward the proximal end of the collar 132. As shown in FIG. 46D, the cutting element 180 may be actuated to rotate about the two pivots 186 to the opposite side of the inner surface 134 of the collar 132 from the side the cutting element 180 was disposed against in the unactuated position. The cutting element 180 may cut or sever tissue as the cutting element 180 pivots between the two sides of the inner surface 134 of the collar 132. In some embodiments, the cutting element 180 may be actuated to pivot back toward the unactuated position to additionally cut or sever tissue.

The collar 132, the cutting element 180, and the pivots 186 may be sized shaped and configured such that the cutting element 180 cuts target tissue at a desired location. For example, the pivots 186 may be positioned a distance from the distal end of the collar 132, the cutting element 180 may be sized or shaped, and the cutting element 180 may be configured to actuate proximally or distally such that the cutting element 180 cuts the target tissue at the desired position.

The cutting element 180 may be actuated by actuation of the cutting actuation wire 310. For example, the cutting element 180 may pivot in a first direction via proximal actuation of the cutting actuation wire 310 and may pivot in a second direction via distal actuation of the cutting actuation wire 310. In some embodiments, the cutting element 180 may be repeatedly actuated in the first and second directions to sufficiently cut or sever the target tissue. In some embodiments, the cutting element 180 pivots 180° about the two pivots 186 when actuated.

In some embodiments, such as any embodiment described herein, the cutting element 180 may be powered or energized to assist in cutting or otherwise severing the target tissue. The cutting element 180 may receive thermal, electrical, actuation, or other energy via the cutting actuation wire 310 to electrocauterize, ablate, or otherwise sever the target tissue. The cutting actuation wire 310 may be coupled with a power source via the actuation assembly 200, as described below, such that the cutting element 180 electrocauterizes the target tissue when the cutting element 180 is actuated. For example, any of the cutting elements 180 described in FIGS. 28-46D may be energized to assist in cutting or otherwise severing the target tissue.

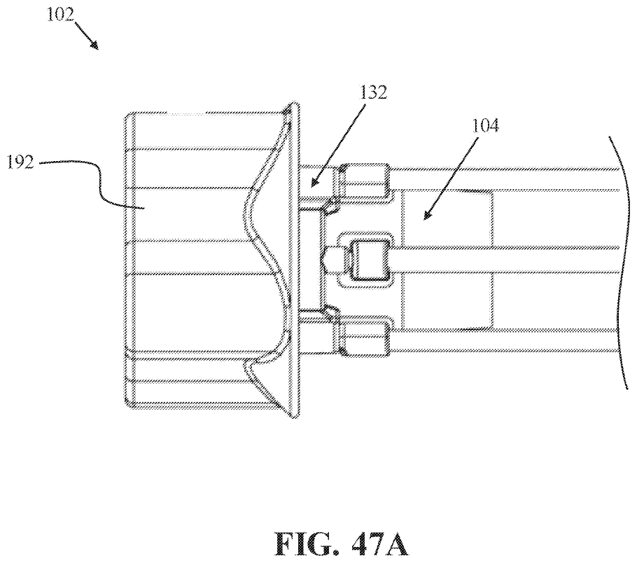

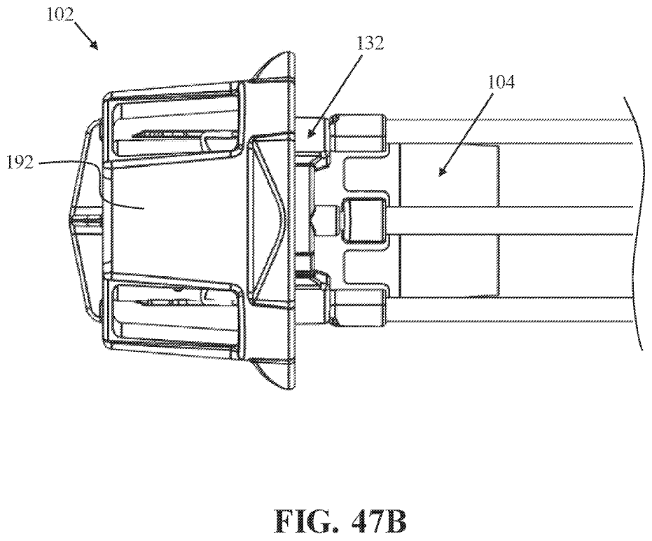

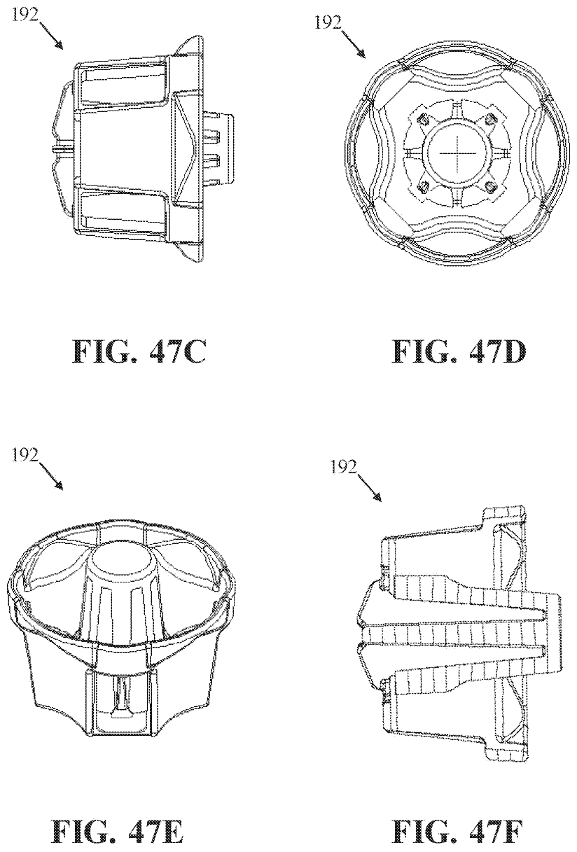

As shown in FIGS. 47A-47F, the device may also include a safety cap 192 configured to prevent premature actuation and deployment of the closure and retrieval assembly 102. The safety cap 192 may be configured to operatively couple with a distal end of the housing 104 such as to protect the distal end of the device. In some embodiments, the device 100 may be assembled and packaged with the safety cap 192 disposed on the distal end of the closure and retrieval assembly 102 to prevent premature actuation and deployment of the closure and retrieval assembly 102 and to protect a user's hands from the components of the closure and retrieval assembly 102.

The safety cap 192 may be configured to be placed around the collar 132 and housing 104 such that the cinch element 170 and the cutting element 180 are encased or otherwise surrounded by the safety cap 192. Additionally, the safety cap 192 may also be sized, shaped, and configured, to maintain the abutment of the collar 132 and the housing 104 before use. For example, when the safety cap 192 is disposed on the distal end of the device 100, the safety cap 192 may prevent the collar 132 from separating from the housing 104 and may prevent actuation of the cinch element 170 and the cutting element 180. In some embodiments, as shown in FIGS. 47B-47F, the safety cap 192 may include one or more apertures through which a user may perform quality checks on the device 100 without removing the safety cap 192, such as to check whether the cinch element 170 is properly disposed on the housing 104. The safety cap 192 may include one or more arms in the middle of the safety cap 192 which are configured to compress when the safety cap 192 is disposed on the housing 104 such that the force of the arms against the housing 104 hold the safety cap 192 onto the housing 104.

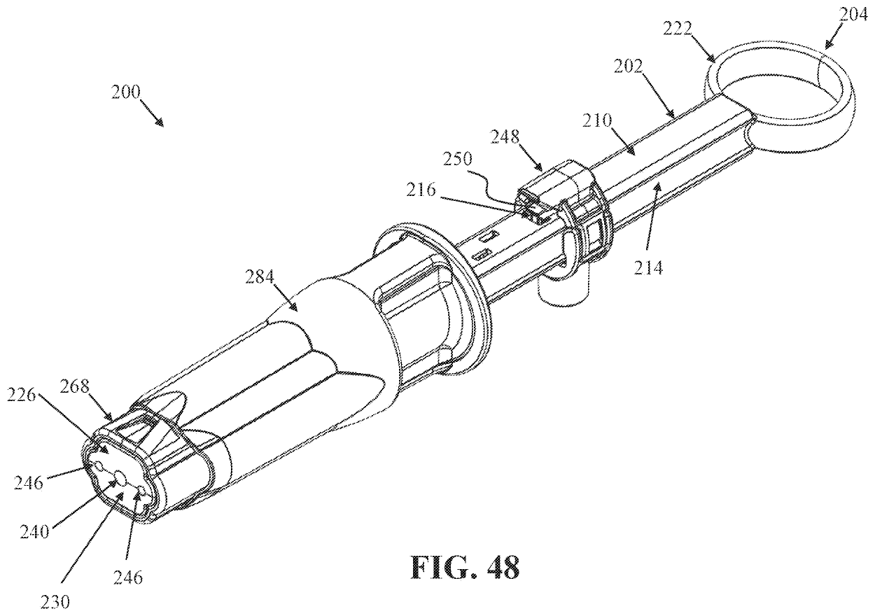

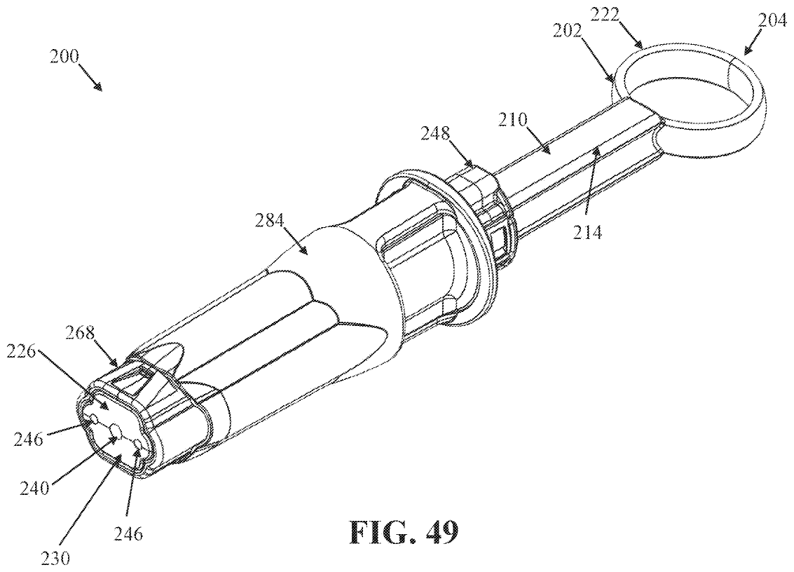

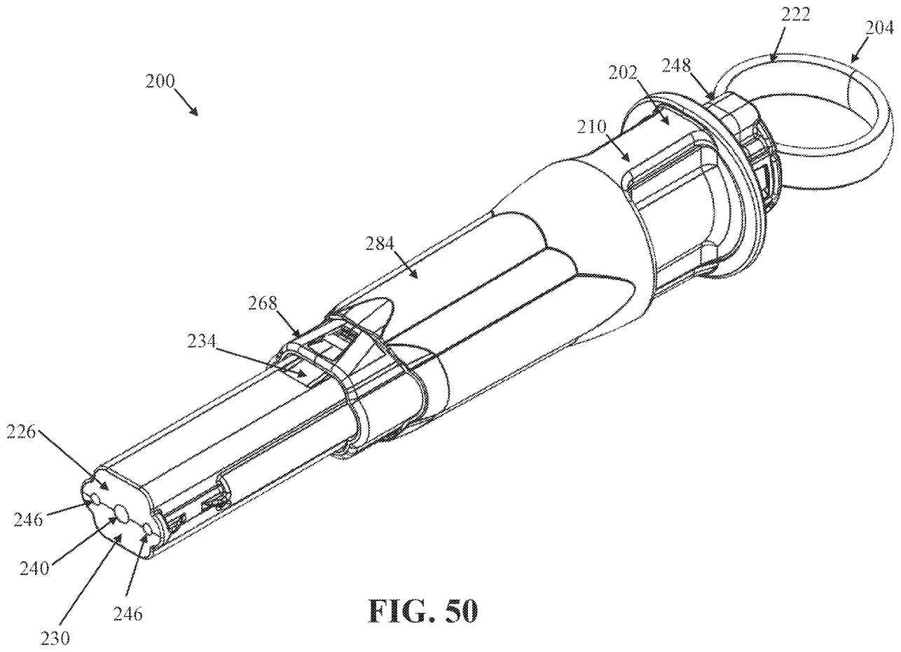

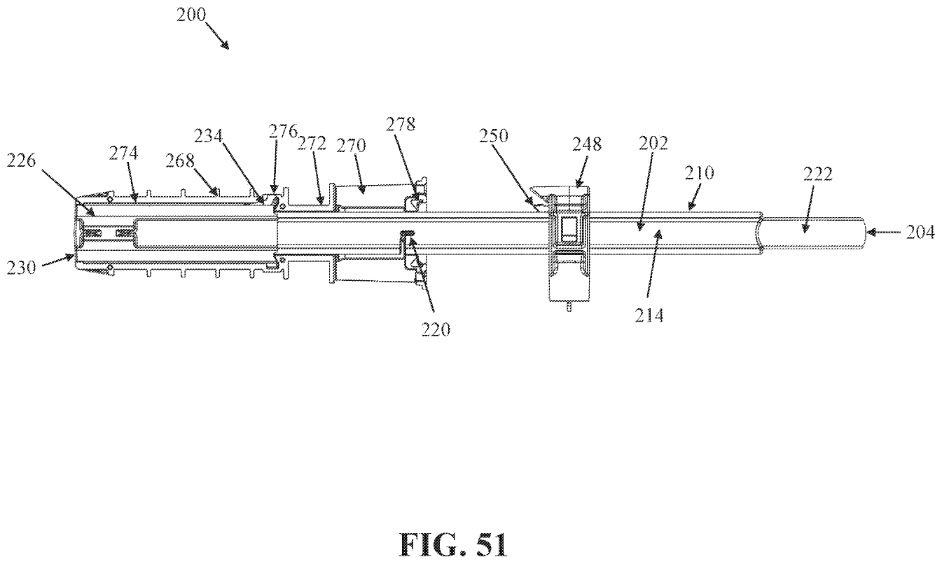

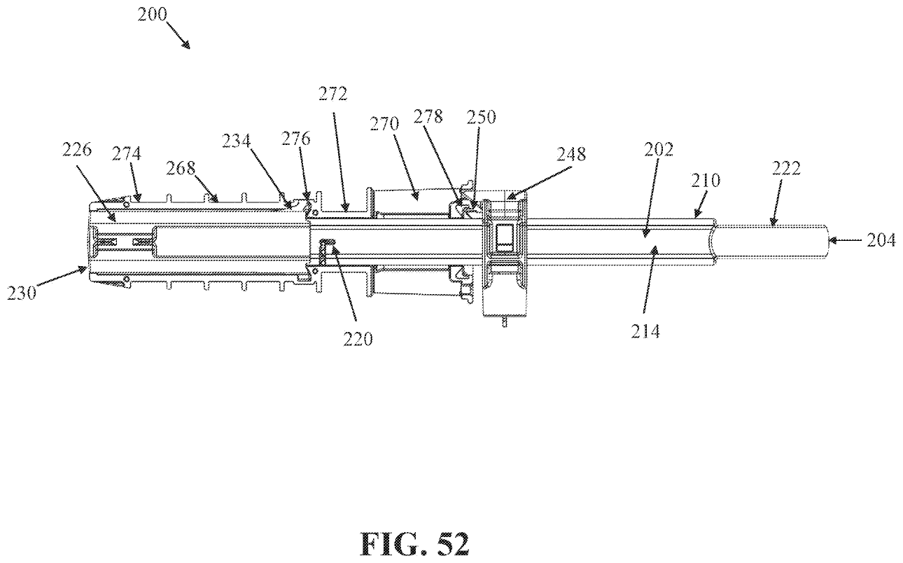

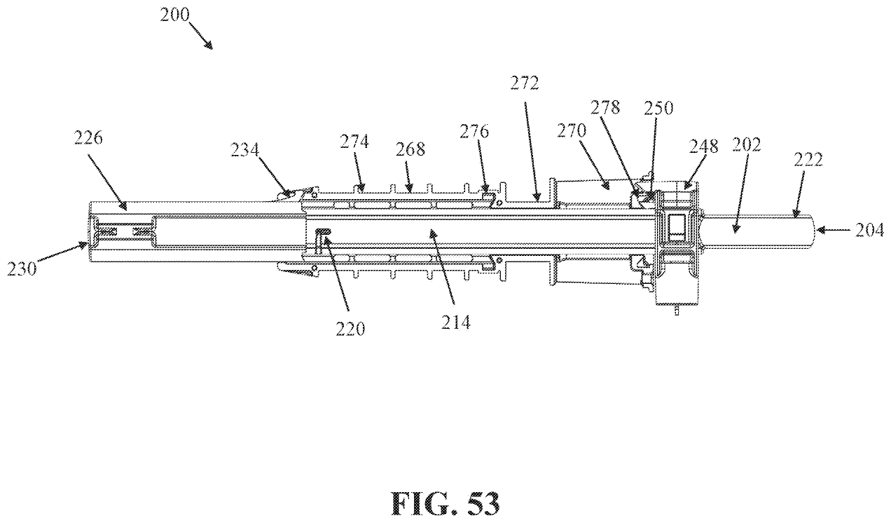

Referring now to FIGS. 48-53, an exemplary actuation assembly 200 is depicted. The actuation assembly 200 may be coupled with a stationary distal component and a movable distal component that is movable relative to the stationary distal component. The stationary distal component may be coupled to a distal end of an endoscope. The actuation assembly 200 may be configured to move the movable distal component distally and proximally relative to the stationary distal component. The actuation assembly 200 may also be coupled to an actuatable element disposed within the movable distal component. The actuation assembly 200 is movable between an unactuated position (FIGS. 48 and 51), a partially actuated position (FIGS. 49 and 52), and a fully actuated position (FIGS. 50 and 53), such as to control the deployment and actuation of the closure and retrieval assembly 102. The actuation assembly 200 may be configured to proximally actuate the actuatable element, such as after the movable distal component has been moved distally relative to the stationary distal component. In some embodiments, the actuation assembly 200 is configured to move the movable distal component distally relative to the stationary distal component and proximally actuate the actuatable element with a single stroke or actuation.

The actuation assembly 200 may be coupled to the closure and retrieval assembly 102, such as via the catheter sheath assembly 300, to control the operation of the closure and retrieval assembly 102. The actuation assembly 200 may be coupled to the closure and retrieval assembly 102 to move the collar 132 distally relative to the housing 104 and thereby deploy the cinch element 170. The actuation assembly 200 may also be coupled to the closure and retrieval assembly 102 to actuate the cutting element 180. In some embodiments, the actuation assembly 200 is configured and coupled to the closure and retrieval assembly 102 such that a single stroke or movement of the actuation assembly 200 may deploy the cinch element 170 and actuate the cutting element 180 of the closure and retrieval assembly 102.

The actuation assembly 200 includes a shaft 202, a connector 226, a spool 268, and a slider 248. The connector 226, the spool 268, and the slider 248 may each be disposed along a length of the shaft 202 and configured to slide along at least a portion of the shaft 202. The connector 226 may be disposed toward a distal end 206 of the shaft 202 and the slider 248 may be disposed toward a proximal end 204 of the shaft 202.

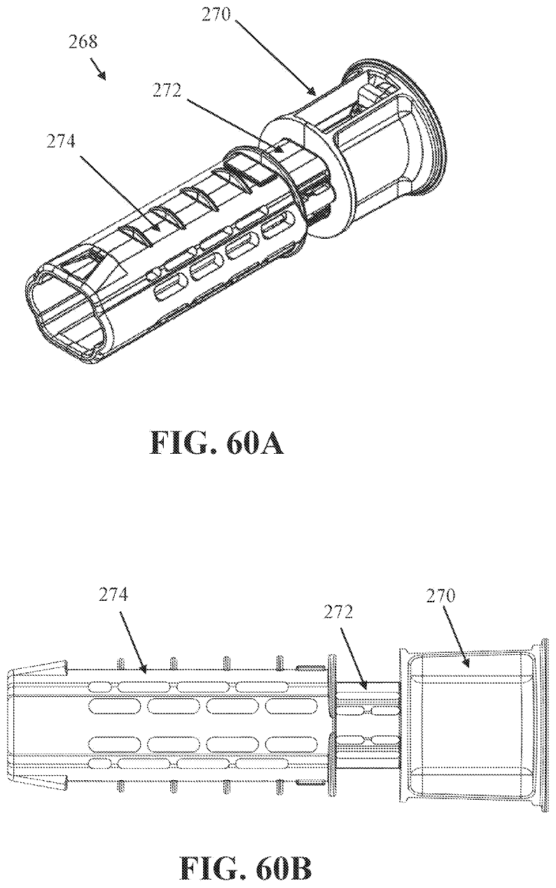

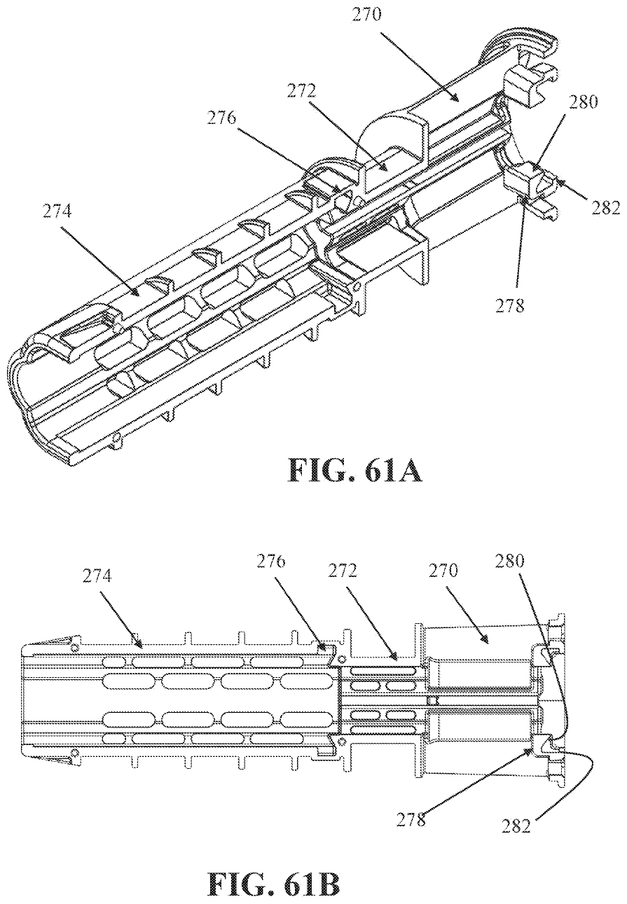

As shown in FIGS. 60A-61B, the spool 268 may include two halves such that the spool 268 may be assembled around the connector 226 and the shaft 202 when the connector 226 is disposed on the shaft 202. In some embodiments, the halves of the spool 268 may be coupled around the shaft 202 and the connector 226 via fasteners, welding, adhesives, or the like. In other embodiments, the halves of the spool 268 are retained in place by an additional component. FIGS. 51-53 show side views of the actuation assembly 200 with one half of the spool 268 removed for illustrative purposes.

As shown in FIGS. 48-50, the actuation assembly 200 may optionally include a spool cover 284. The spool cover 284 may be configured for a user to grasp during operation. The spool cover 284 may be configured to at least partially surround the spool 268. In some embodiments, the spool cover 284 is configured to retain the halves of the spool 268 together when the spool cover 284 is disposed around the spool 268.

As shown in FIGS. 48-54B, the shaft 202 extends longitudinally between the proximal end 204 and the distal end 206. The connector 226 is slidably disposed at least partially on the shaft 202 near the distal end 206 of the shaft 202. The connector 226 may be configured such that a portion of the connector 226 may extend distally to the distal end 206 of the shaft 202 when the connector 226 is disposed on the shaft 202. The slider 248 is slidably disposed on the shaft 202 proximally from the connector 226, such as toward the proximal end 204 of the shaft 202. The spool 268 may be slidably disposed on the shaft 202 and may at least partially surround the connector 226.

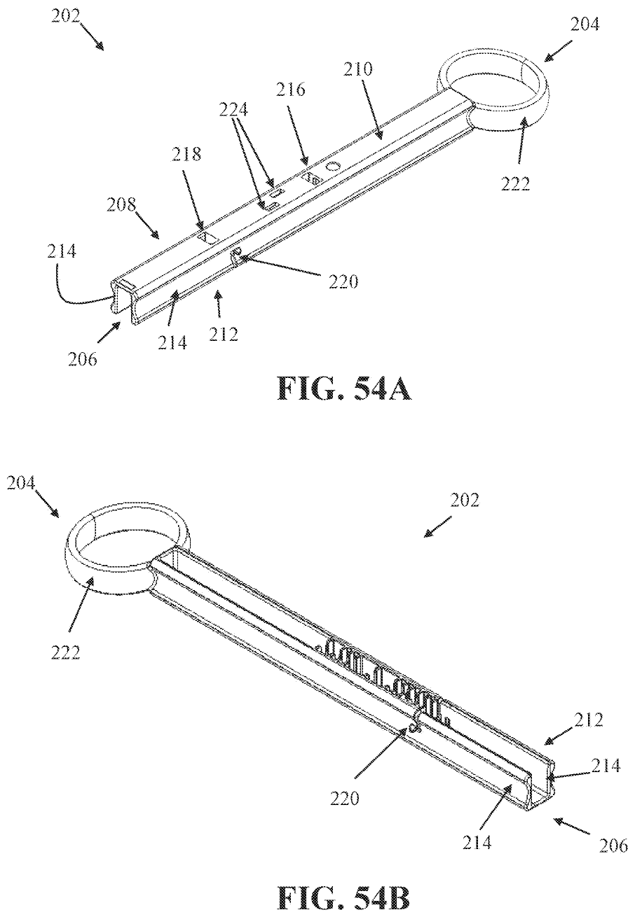

As shown in FIGS. 54A and 54B, the shaft 202 may be substantially U-shaped with a top portion 208 having a top surface 210 and a bottom portion 212 having two sides 214 extending downwardly from an underside of the top portion 208. The top surface 210 may be substantially planar. The sides 214 may be rounded or curved inwardly, such as to retain the connector 226, the slider 248, and the spool 268 on the shaft 202 during operation of the actuation assembly 200, as described below. The sides 214 may be spaced apart from one another such that a channel or groove extends along the bottom portion 212 of the shaft 202 between the sides 214.

The top surface 210 may include a slider coupling portion 216 configured to operably couple with the slider 248. The slider 248 may remain longitudinally in place relative to the shaft 202 when the slider 248 is coupled with the slider coupling portion 216, as described below. The slider coupling portion 216 may be an aperture, bore, detent, or the like extending into or through the top surface 210 and configured to receive a downwardly extending portion of the slider 248. In some embodiments, the slider coupling portion 216 is a substantially rectangular aperture extending through the top surface 210 of the shaft 202. The slider coupling portion 216 may be spaced a distance from the distal end 206 of the shaft 202 corresponding to the distance the cutting element 180 moves relative to the housing 104 when the closure and retrieval assembly 102 is moved from the undeployed configuration to the deployed configuration and before the cutting element 180 is actuated, as described below.

The top surface 210 may include a connector coupling portion 218 configured to operably couple with the connector 226. The connector 226 may remain longitudinally in place relative to the shaft 202 when the connector 226 is coupled with the connector coupling portion 218, as described below. The connector coupling portion 218 may be an aperture, bore, detent, or the like extending into or through the top surface 210 and configured to receive a downwardly extending portion of the connector 226. In some embodiments, the connector coupling portion 218 is a substantially rectangular aperture extending through the top surface 210 of the shaft 202. The connector coupling portion 218 may be spaced a distance from the distal end 206 of the shaft 202 corresponding to the distance the collar 132 moves relative to the housing 104 when the closure and retrieval assembly 102 is moved from the undeployed configuration to the deployed configuration, as described below.

Each side 214 of the shaft 202 may include a wire receiving portion 220 configured to retain a proximal end of an actuation wire, catheter, or the like. Each wire receiving portion 220 may be sized, shaped, and configured such that a proximal end of one of the deployment actuation wires 304 may be inserted into the wire receiving portion 220 and operably secured in the wire receiving portion 220, as described below. When the proximal end of the one of the deployment actuation wires 304 is disposed in the wire receiving portion 220, the position of the deployment actuation wire 304 may be substantially fixed in the longitudinal direction in relation to the wire receiving portion 220. For example, the deployment actuation wires 304 received in the wire receiving portions 220 may move distally with distal movement of the shaft 202 and may move proximally with proximal movement of the shaft 202. In some embodiments, the wire receiving portions 220 are spaced from the distal end 206 of the shaft 202 a similar distance as the connector coupling portion 218. While the wire receiving portions 220 have been described as operably securing the proximal end of the deployment actuation wires 304, it will be understood that the wire receiving portions 220 may be sized, shaped, and configured to secure the proximal end of any suitable actuation element, such as a catheter, a suture, a tether, or the like.

In the illustrated embodiment, each wire receiving portion 220 is substantially J-shaped with an inlet portion extending upwardly from the bottom of the respective side 214, a neck portion extending laterally from an upper end of the inlet portion, and a retaining portion disposed opposite the neck from the inlet portion. The proximal end of one of the deployment actuation wires 304 may be inserted up through the inlet portion, laterally across the neck portion, and be operably received in the retaining portion of the wire receiving portion 220. The neck portion may have a width less than the width of the inlet portion and the retaining portion to secure the proximal end of the deployment actuation wire 304 in the retaining portion of the wire receiving portion 220.

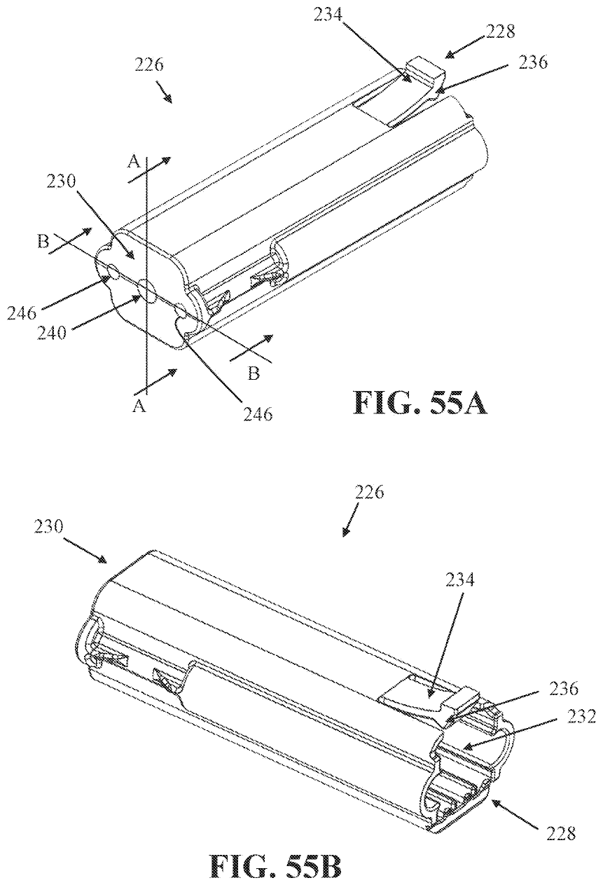

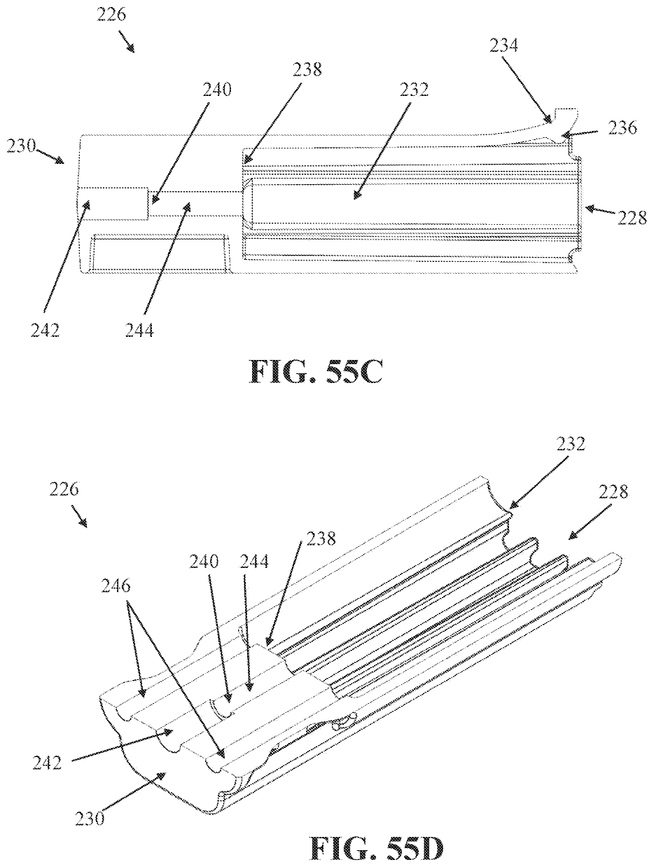

As shown in FIGS. 55A-55D, the connector 226 is substantially cylindrical with a proximal end 228 and a distal end 230 opposite the proximal end 228. The distal end 230 may be substantially planar. The connector 226 may also include a shaft receiving portion 232 extending toward the distal end 230 from the proximal end 228 of the connector 226. The shaft receiving portion 232 may be substantially hollow. The shaft receiving portion 232 may be configured to receive the distal end 206 of the shaft 202 such that the distal end 206 of the shaft 202 may slide within the shaft receiving portion 232.

The connector 226 also includes a projection 234 near the proximal end 228. The projection 234 may pivot upwardly from an upper portion of the connector 226 (e.g., the top of the connector 226 in FIGS. 55A-55C) and proximally toward the proximal end 228 of the connector 226. The projection 234 includes a flange 236 at the proximal end of the projection 234 that extends downwardly from the remainder of the projection 234. The flange 236 may be sized, shaped, and configured to operatively couple with the connector coupling portion 218 of the shaft 202, as described below. In some embodiments, the projection 234 is biased upwardly from the remainder of the connector 226. For example, the projection 234 may be sized, shaped, and configured to bias upwardly and/or the projection 234 may be biased upwardly when the flange 236 slides along the top surface 210 of the shaft 202 outside of the connector coupling portion 218.

The connector 226 may also include a shoulder 238 at a distal end of the shaft receiving portion 232. The shoulder 238 may be configured to abut the distal end 206 of the shaft 202 at a distal end of the shaft receiving portion 232 to prevent the connector 226 from sliding farther proximally over the shaft 202.

The connector 226 includes one or more first bores 240 extending through a central portion of the connector 226 from the distal end 230 to the shoulder 238. In the illustrated embodiment, the connector 226 includes one first bore 240. However, it will be understood that the connector 226 may include any suitable numbers of first bores 240. For example, the connector 226 may include a number of first bores 240 corresponding to the number of cutting actuation wires 310 of the device 100.

The first bore 240 may be disposed through the connector 226 such that the first bore 240 is positioned between the sides 214 of the shaft 202 when the shaft 202 is disposed in the shaft receiving portion 232. The first bore 240 may be substantially circular configured to receive the housing catheter 306 and the collar catheter 308 therethrough. For example, the first bore 240 may have a diameter slightly larger than the outer surface of the housing catheter 306. The connector 226 may be configured to receive the housing catheter 306, the collar catheter 308, and the cutting actuation wire 310 therethrough such that the collar catheter 308 and the cutting actuation wire 310 extends proximally through the shaft receiving portion 232, as described below

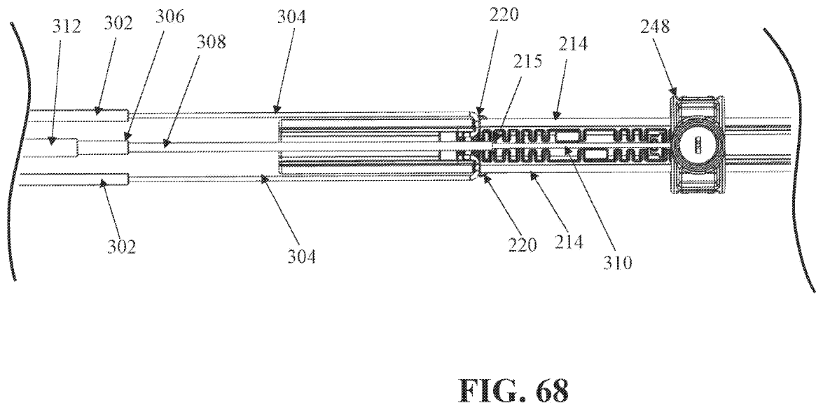

In some embodiments, the catheter sheath assembly 300 includes a strain relief catheter 312 (FIG. 68) disposed around the housing catheter 306. The strain relief catheter 312 may have an inner diameter larger than the housing catheter 306 such that the strain relief catheter 312 may be disposed around at least a proximal portion of the housing catheter 306. The strain relief catheter 312 may be operable to reduce the stress and/or strain of the housing catheter 306 during operation, such as to prevent the housing catheter 306 from buckling during operation. The strain relief catheter 312 may comprise HDPE, PTFE, shape memory metal, such as Nitinol, stainless steel, or the like, or combinations thereof. In a preferred embodiment, the strain relief catheter 312 comprises HDPE.

The first bore 240 of the connector 226 may have a first portion 242 at a distal end and a second portion 244 at a proximal end. The first and second portions 242, 244 may be substantially circular with the second portion 244 having a diameter less than a diameter of the first portion 242. The first portion 242 may be sized to receive the strain relief catheter 312, the housing catheter 306, and the collar catheter 308. The second portion 244 may be sized to receive the housing catheter 306 and the collar catheter 308 therethrough and to prevent the strain relief catheter 312 from extending proximally beyond the first portion 242 of the connector 226. The proximal end of the strain relief catheter 312 may be secured in the first portion 242 of the first bore 240.

The connector 226 may also include one or more second bores 246 extending through lateral portions of the connector 226 from the distal end 230 to the shoulder 238. The second bores 246 may be disposed through the connector 226 such that the second bores 246 are disposed laterally beyond the sides 214 of the shaft 202 when the shaft 202 is disposed in the shaft receiving portion 232. Each second bore 246 may be substantially circular and be sized to receive the clip deployment catheter 302 therethrough. For example, the connector 226 may be configured to receive one of the clip deployment catheters 302 through each of the second bores 246 such that the deployment actuation wires 304 extend proximally through the shaft receiving portion 232, as described below. In the illustrated embodiment, the connector 226 has two second bores 246. However, it will be understood that the connector 226 may include any suitable number of second bores 246. For example, the connector 226 may have a number of second bores 246 corresponding to the number of deployment actuation wires 304 used with the device 100.

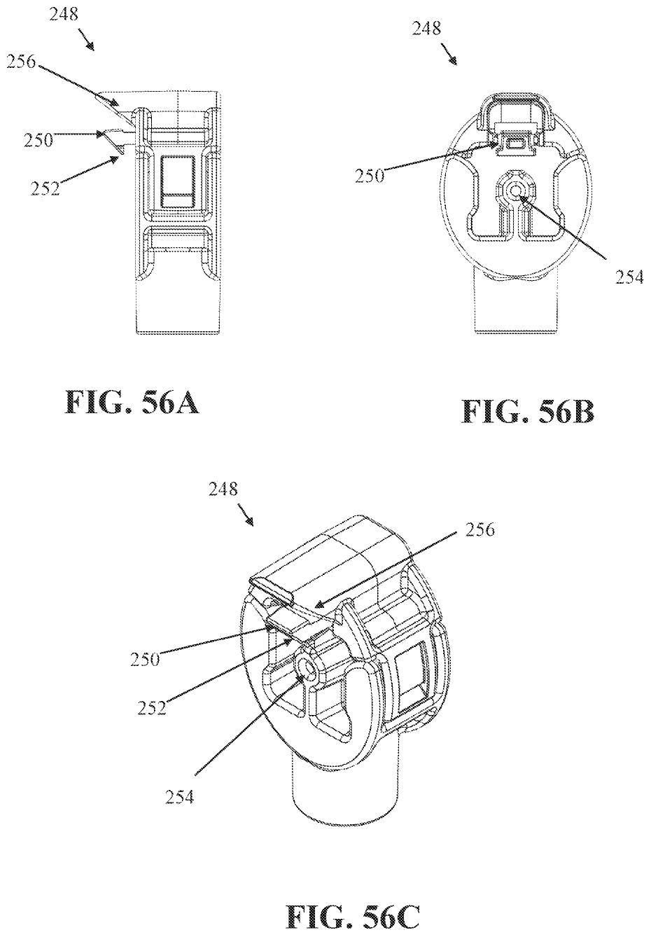

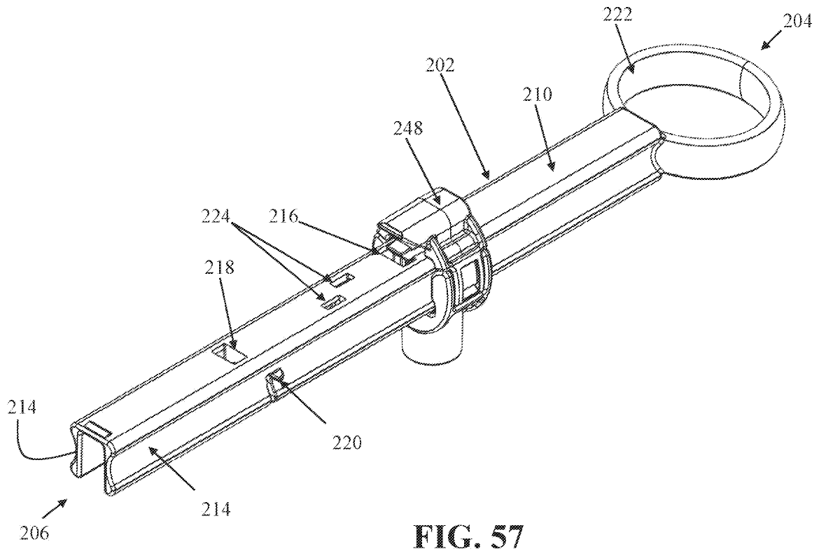

As shown in FIGS. 48-53 and 56A-57 the slider 248 may be disposed substantially around the shaft 202 at a location proximal to the proximal end 228 of the connector 226. The slider 248 may be configured to control the actuation of the cutting element 180 during actuation of the actuation assembly 200. The slider 248 may include a coupler 250 extending distally and configured to operatively couple with shaft 202, such as with the slider coupling portion 216 of the shaft 202. The coupler 250 includes a flange 252 at a distal end of the coupler 250 that extends downwardly from the remainder of the coupler 250. The flange 252 may be sized, shaped, and configured to operatively be disposed in the slider coupling portion 216 of the shaft 202 to couple the slider 248 to the shaft 202, as described below. The slider 248 may remain substantially in position relative to the shaft 202 when the flange 252 is disposed in the slider coupling portion 216. The slider 248 may slide proximally and/or distally along a length of the shaft 202 when the coupler 250 is decoupled from the slider coupling portion 216. The coupler 250 may also be configured to operatively couple the slider 248 with the spool 268, as described below.

The slider 248 may also include an actuation wire receiving portion 254. The actuation wire receiving portion 254 may be configured to receive and retain a proximal end of the cutting actuation wire 310. The actuation wire receiving portion 254 may be configured to retain the proximal end of the cutting actuation wire 310 such that the cutting actuation wire 310 moves in concert with the slider 248. For example, the cutting actuation wire 310 may move distally with distal movement of the slider 248 and may move proximally with proximal movement of the slider 248. The actuation wire receiving portion 254 may be disposed in a central portion of the slider 248 such that the actuation wire receiving portion 254 is below the top surface 210 of the shaft 202 and between the sides 214 of the shaft 202 when the slider 248 is disposed on the shaft 202.

In some embodiments, the slider 248 includes an angled shoulder 256 extending proximally from a top portion of the slider 248 above the coupler 250. The angled shoulder 256 may be sized, shaped, and configured to retain the coupling between the slider 248 and the spool 268 when the coupler 250 of the slider 248 is coupled with the spool 268. For example, the angled shoulder 256 may prevent the proximal end of the spool 268 from sliding proximally over the slider 248 and may bias the proximal end of the spool 268 downwardly to maintain the coupling between the coupler 250 of the slider 248 and the spool 268.

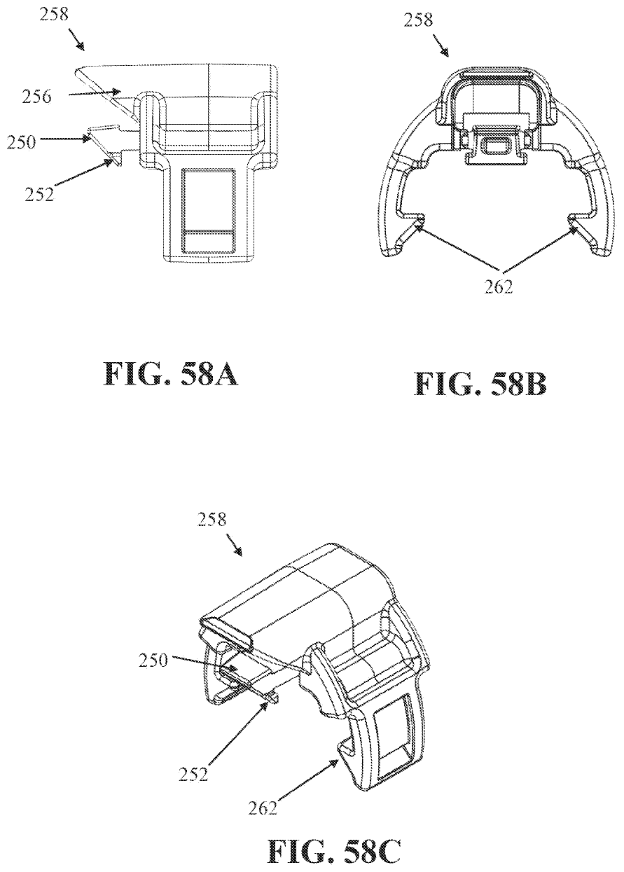

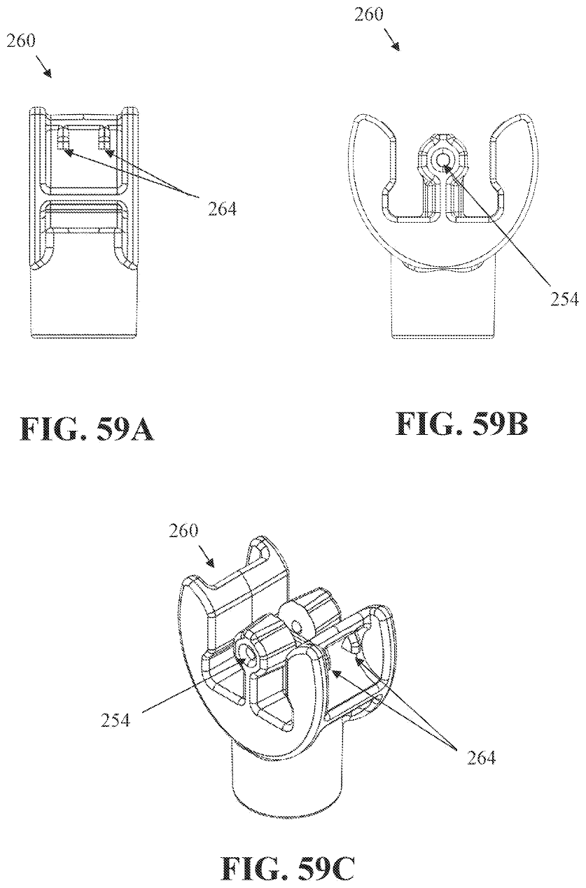

In some embodiments, as shown in FIGS. 58A-59C, the slider 248 includes an upper portion 258 and a lower portion 260. The upper portion 258 is configured to be disposed around the top portion 208 of the shaft 202 and the lower portion 260 is configured to be disposed around a bottom portion 212 of the shaft 202. In some embodiments, the upper and lower portions 258, 260 are snap-or press-fit together. For example, the upper portion 258 may have inwardly extending projections 262 and the lower portion 260 may have corresponding ledges or abutments 264. The upper and lower portions 258, 260 may be disposed around the shaft 202 and the upper portion 258 may be pressed downwardly with respect to the lower portion 260 such that the projections 262 of the upper portion 258 snap fit around the abutments 264 of the lower portion 260, thereby securing the upper and lower portions 258, 260 around the shaft 202.

As shown in FIGS. 51-53 and 60A-61B, the spool 268 may be disposed along a length of the shaft 202 and may be partially disposed around the connector 226. The spool 268 may be configured to deployment of the closure and retrieval assembly 102 and/or to actuate the cutting element 180 during actuation of the actuation assembly 200. The spool 268 includes a proximal portion 270, a medial portion 272, and a distal portion 274.

The distal portion 274 may be configured to be at least partially disposed around the outside of the connector 226. For example, the distal portion 274 may be substantially tubular with an inner diameter substantially similar to the diameter of the outside of the connector 226. The distal portion 274 may include a projection receiving portion 276 at a proximal end of the distal portion 274. The projection receiving portion 276 may be sized, shaped, and configured to operatively receive the projection 234 of the connector 226 when the projection 234 is flexed upwardly. The distal portion 274, such as the projection receiving portion 276, may be configured to operatively coupled with the connector 226 when the projection 234 is flexed upwardly such that the connector 226 moves proximally and distally with the spool 268. For example, the proximal and distal sides of the projection receiving portion 276 may be configured to abut the projection 234 when the projection 234 is flexed upwardly such that the connector 226 moves with the spool 268.

The radially inner surface of the distal portion 274 may also be configured to slide over the connector 226, including the projection 234, when the projection 234 of the connector 226 is flexed downwardly, such as when the flange 236 of the projection 234 is disposed in the connector coupling portion 218 of the shaft 202. For example, the radially inner surface of the distal portion 274 may have a diameter larger than the outside diameter of the connector 226 when the flange 236 of the projection 234 is disposed in the connector coupling portion 218 of the shaft 202 such that the distal portion 274 may slide proximally over the connector 226.

The medial portion 272 may be configured to prevent the connector 226 from moving proximally past the medial portion 272 and/or to prevent the medial portion 272 from moving distally past the connector 226. For example, the medial portion 272 may be substantially tubular with an inner diameter less than the outer diameter of the connector 226. In some embodiments, the outer surface of the medial portion 272 has a diameter less than the diameters of the outer surfaces of the proximal and distal portions 270, 274 such that a user may grasp the spool 268 around the outside of the medial portion 272 during operation.

The proximal portion 270 includes a slider coupler receiver 278 at the proximal end of the proximal portion 270. The slider coupler receiver 278 may be configured to decouple the coupler 250 of the slider 248 from the slider coupling portion 216 of the shaft 202 and to couple with the spool 268 with the slider 248. The slider coupler receiver 278 may include a ramp 280 configured to lift the flange 252 of the coupler 250 out of the slider coupling portion 216 of the shaft 202. The slider coupler receiver 278 may also include a proximal shoulder 282 configured to retain the coupler 250 of the slider 248 in the slider coupler receiver 278. When the spool 268 is moved proximally toward the slider 248 such that the proximal end of the proximal portion 270 contacts the slider 248, the slider coupler receiver 278, such as the ramp 280 of the slider coupler receiver 278, may contact a bottom surface of the flange 252 of the coupler 250 to lift the coupler 250 out of engagement with the slider coupling portion 216 of the shaft 202. Continued relative proximal movement of the spool 268 may move the distal end of the coupler 250 into the slider coupler receiver 278. The proximal shoulder 282 may abut a proximal portion of the coupler 250 to prevent or otherwise restrict the coupler 250 from disengaging with the slider coupler receiver 278. The slider 248 may move in concert with the spool 268 when the coupler 250 is retained within the slider coupler receiver 278.

In some embodiments, as shown in FIGS. 54A and 54B, the shaft 202 also includes a ring 222 disposed at the distal end of the shaft 202. The ring 222 may be sized, shaped, and configured for a user to grasp and control during use. The ring 222 may be perpendicular to the top surface 210 of the shaft 202. The ring 222 may be sized, shaped, and configured to retain the slider 248 on the shaft 202, such as by preventing the slider 248 from sliding proximally past the ring 222. For example, an outer diameter of the ring 222 may be larger than the inner diameter of the slider 248 such that the slider 248 is prevented from sliding proximally over the ring 222.

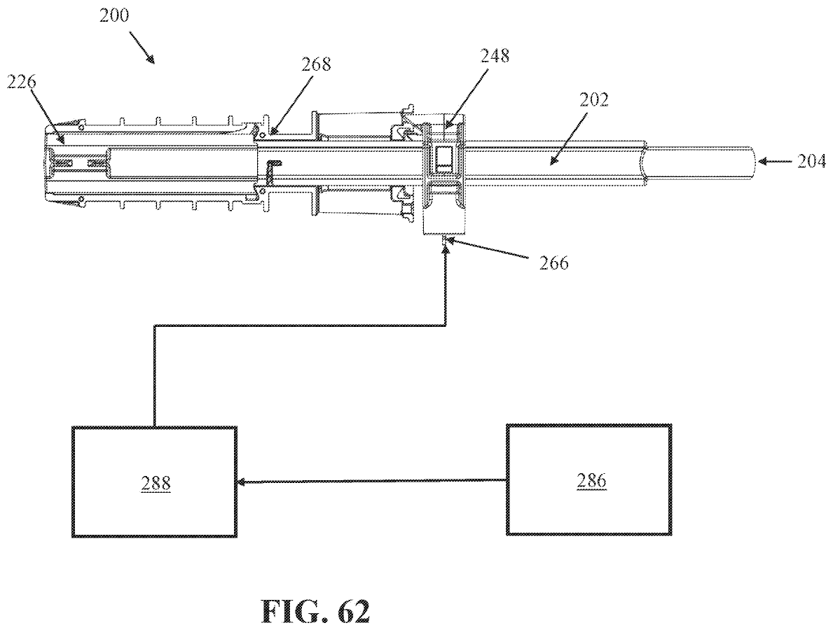

In some embodiments, as shown in FIG. 62, the slider 248 may include an energy connector 266 configured to receive energy, such as thermal, electrical, mechanical, or other energy, such as to energize the cutting element 180. The energy connector 266 may be coupled to an energy source 286, such as via one or more wires, to receive energy. The energy source 286 may be batteries, battery packs, power banks, or electric generators, connections to power outlets or sources, or any combination thereof. The energy connector 266 may be connected to the actuation wire receiving portion 254, such as to direct energy to the cutting actuation wire 310 and the cutting element 180.

In some embodiments, the actuation assembly 200 includes a switch 288 configured to direct or otherwise control the flow of energy from the energy source 286 to the energy connector 266. The switch 288 may be configured to prevent the flow of energy from the energy source 286 to the energy connector 266 in an unactuated position and to allow the flow of energy from the energy source 286 to the energy connector 266 in the actuated position. In some embodiments, the switch 288 is a foot pedal such that a user may operate the actuation assembly 200 with both hands and simultaneously control the flow of energy to the energy connector 266.

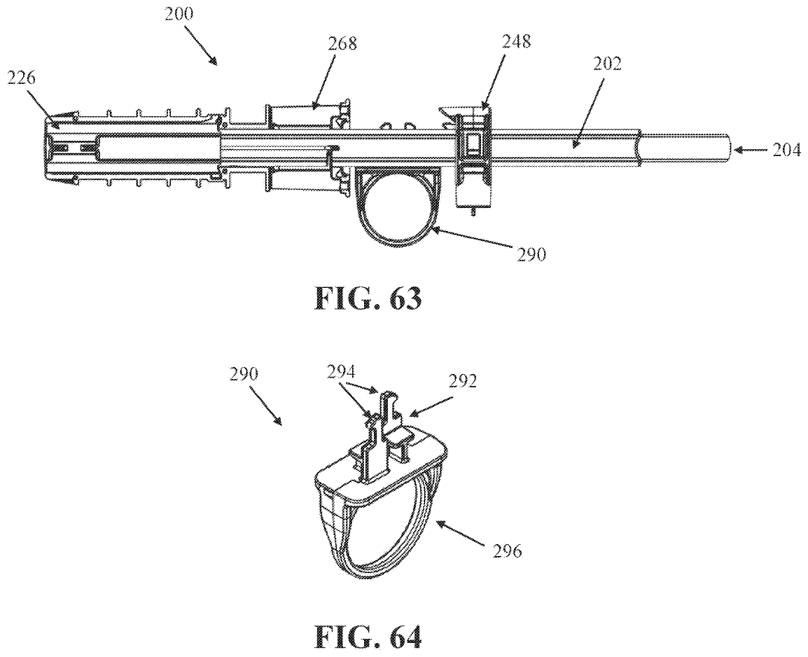

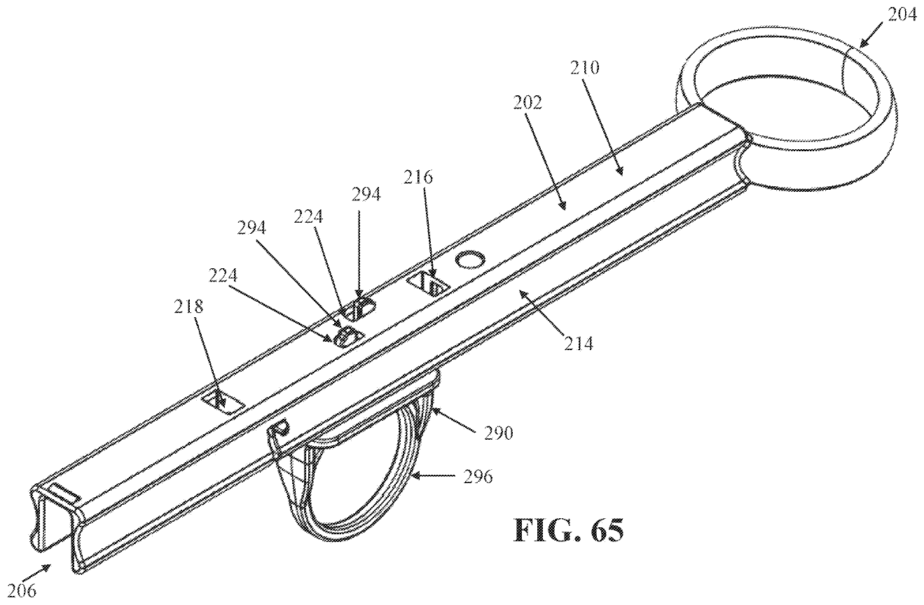

Referring now to FIGS. 63-65, the actuation assembly 200 may also include a stopper 290 disposed along a length of the shaft 202. The stopper 290 may configured to prevent premature actuation of the actuation assembly 200. The stopper 290 may be disposed between the proximal portion 270 of the spool 268 and the coupler 250 of the slider 248 when the actuation assembly 200 is in the unactuated position to prevent the connector 226, spool 268, and slider 248 from sliding along the shaft 202.

The stopper 290 may include a coupling portion 292 configured to couple with the shaft 202. In the illustrated embodiment, the coupling portion 292 includes two prongs 294 configured to extend through and be operatively received by stopper receiving apertures 224 in the shaft 202. The prongs 294 may each extend upwardly from the remainder of the stopper 290 and have an outwardly extending flange which secures the prong 294 in one of the stopper receiving apertures 224.

The coupling portion 292 of the stopper 290 may be configured such that the prongs 294 are biased outwardly such that the flanges retain the prongs 294 in the stopper receiving apertures 224. The prongs 294 may be biased inwardly, such as by squeezing the prongs 294 together, and inserted through the stopper receiving apertures 224. After the prongs 294 are inserted through the stopper receiving apertures 224 such that the flanges of the prongs 294 are disposed on the opposite side of the shaft 202 from the remainder of the stopper 290, the biasing force may be released such that the flanges of the prongs 294 bias outwardly beyond the outer edges of the stopper receiving apertures 224, thereby securing the stopper 290 with the shaft 202. When the stopper 290 is coupled to the shaft 202 the stopper 290 may prevent movement of the connector 226, the spool 268, and/or the slider 248 relative to the shaft 202.

When the device 100 is ready for use, a user may pinch the prongs 294 of the stopper 290 together and remove the prongs 294 from the stopper receiving apertures 224 to decouple the stopper 290 from the shaft 202. The stopper 290 may also including a grasping portion 296 configured to be grabbed by a user, such as to disconnect the stopper 290 from the shaft 202 when the device 100 is ready to be used. In the illustrated embodiment, the grasping portion 296 is a ring. However, it will be understood that the grasping portion 296 may have any size, shape, and configuration such that a user may grab the stopper 290.

Actuation of the actuation assembly 200 will now be described with reference to FIGS. 48-53. The actuation assembly 200 may be moved from an unactuated position (FIGS. 48 and 51) to a partially actuated position (FIGS. 49 and 52), and to a fully actuated position (FIGS. 50 and 53).

As shown in FIG. 51, when the actuation assembly 200 is in the unactuated position, the distal end 206 of the shaft 202 is disposed in the shaft receiving portion 232 of the connector 226 and spaced apart from the shoulder 238 of the connector 226. The spool 268 is disposed around the connector 226 and the shaft 202. The distal portion 274 of the spool 268 is disposed substantially around the connector 226. The proximal portion 270 of the spool 268 is spaced distally apart from the slider 248. The projection 234 of the connector 226 is disposed in the projection receiving portion 276 of the spool 268 such that the connector 226 and the spool 268 are coupled. The flange 236 of the projection 234 may be disposed above the top surface 210 of the shaft 202. The spool cover 284 may be disposed around the outside of the spool 268.

The slider 248 is disposed toward the proximal end 204 of the shaft 202 from the connector 226 and the spool 268. The coupler 250 of the slider 248 may be coupled with the shaft 202 such that the position of the slider 248 is longitudinally fixed relative to the shaft 202. For example, the flange 252 of the coupler 250 is disposed in the slider coupling portion 216.

As shown in FIG. 52, when the actuation assembly 200 is in the partially actuated position, the shaft 202 is moved distally relative to the connector 226 and the spool 268 such that the spool 268 may decouple from the connector 226. For example, a user may hold the spool 268 relatively stationary, such as by grasping the spool cover 284, and push or otherwise move the shaft 202 distally, such as by grasping the ring 222 and pushing the ring 222 toward the spool 268.

During initial movement of the shaft 202 from the unactuated position, the projection 234 of the connector 226 may be disposed in the projection receiving portion 276 of the spool 268 such that the position of the connector 226 is relatively fixed in relation to the position of the spool 268. The flange 236 of the connector 226 may be disposed above the top surface 210 of the shaft 202 such that the shaft 202 may move distally through the spool 268 and the connector 226, such as distally into the shaft receiving portion 232 of the connector 226. Additionally, the coupler 250 of the slider 248 may be coupled with the slider coupling portion 216 of the shaft 202 such that the slider 248 may move distally in concert with the shaft 202.

The shaft 202 may be moved distally relative to the spool 268 until the projection 234 of the connector 226 couples with the connector coupling portion 218 of the shaft 202. The shaft 202 may be moved distally relative to the spool 268 and the connector 226 until the flange 236 of the connector 226 is disposed substantially above the connector coupling portion 218 of the shaft 202. The projection 234 may flex downwardly from the projection receiving portion 276 such that the flange 236 of the connector 226 is coupled with or otherwise disposed in the connector coupling portion 218. The downward movement of the projection 234 may decouple the spool 268 from the connector 226 such that the that the spool 268 may move proximally relative to the shaft 202 and the connector 226, such as when the shaft 202 is moved farther distally.

In some embodiments, the distal end 206 of the shaft 202 abuts the shoulder 238 of the connector 226 when the flange 236 of the connector 226 is disposed in the connector coupling portion 218 of the shaft 202. The abutment between the shoulder 238 and the distal end 206 of the shaft 202 may prevent further distal movement of the shaft 202 relative to the connector 226.

The slider 248 may remain coupled with the slider coupling portion 216 of the shaft 202 such that the slider 248 is moved toward the proximal portion 270 of the spool 268. For example, the flange 252 of the coupler 250 of the slider 248 may be disposed in the slider coupling portion 216 of the shaft 202 such that the slider 248 moves distally in concert with the shaft 202. In some embodiments, the coupler 250 of the slider 248 is disposed near the slider coupler receiver 278 of the spool 268 when the actuation assembly 200 is in the partially actuated position.

When the actuation assembly 200 is moved to the partially actuated position, the movement of the shaft 202 relative to the connector 226 may deploy the closure and retrieval assembly 102. For example, the proximal ends of the clip deployment catheters 302 may be coupled with the distal end 230 of the connector 226 and the proximal ends of the deployment actuation wires 304 may be coupled with the wire receiving portions 220 of the shaft 202. As the shaft 202 is moved distally into the shaft receiving portion 232 of the connector 226, the distance between the distal end 230 of the connector 226 and the wire receiving portions 220 of the shaft 202 is decreased. The distal movement of the wire receiving portions 220 relative to the distal end 230 of the connector 226 may extend the deployment actuation wires 304 distally relative to the clip deployment catheters 302 thereby deploying the closure and retrieval assembly 102, as described below. In some embodiments, the connector 226 is configured such that the distance between the distal end 230 and the shoulder 238 substantially corresponds to the deployment distance of the closure and retrieval assembly 102.

As shown in FIG. 53, when the actuation assembly 200 is in the fully actuated position, the spool 268 and the slider 248 are moved proximally relative to the shaft 202 and the connector 226. For example, a user may continue to the hold the spool 268 relatively stationary, such as by grasping the spool cover 284, and continue to push or otherwise move the shaft 202 distally, such as by grasping the ring 222 and continuing to push the ring 222 toward the spool 268. The spool 268 and the slider 248 may be moved to a position near the proximal end 204 of the shaft 202. In some embodiments, the spool 268 and the slider 248 move proximally relative to the shaft 202 until the proximal side of the slider 248 abuts the ring 222 of the shaft 202. In such a position, the distance between the slider 248 and the distal end 230 of the connector 226 is maximized.

During initial distal movement of the shaft 202 from the partially actuated position, the spool 268 may move proximally relative to the shaft 202 until the proximal portion 270 of the spool 268 contacts the slider 248. Upon contact of the proximal portion 270 of the spool 268 with the slider 248, the slider coupler receiver 278 of the spool 268 may contact a bottom surface of the coupler 250 to lift the coupler 250 of the slider 248 out of the slider coupling portion 216 of the shaft 202. For example, the ramp 280 of the slider coupler receiver 278 may contact the flange 252 of the coupler 250 and move proximally relative to the flange 252 to lift the flange 252 out of the slider coupling portion 216. The ramp 280 may lift the flange 252 such that the coupler 250 of the slider 248 is coupled with the slider coupler receiver 278, such as with the coupler 250 retained in the slider coupler receiver 278 via the proximal shoulder 282 of the spool 268. The slider 248 may be decoupled from the shaft 202 and move in concert with the spool 268 when the coupler 250 of the slider 248 is decoupled from the slider coupling portion 216 and coupled with the slider coupler receiver 278 of the spool 268.

The connector 226 may remained coupled with the shaft 202 such that the connector 226 is moved distally relative to the spool 268 and the slider 248. For example, the projection 234 of the connector 226 may remain coupled with the connector coupling portion 218 of the shaft 202 and/or the distal end 206 of the shaft 202 may continue to abut the shoulder 238 of the connector 226.

When the actuation assembly 200 is moved to the fully actuated position, the movement of the slider 248 relative to the shaft 202 and/or the connector 226 may actuate the closure and retrieval assembly 102, such as the actuating the cutting element 180. For example, the actuation wire receiving portion 254 of the slider 248 may be coupled with the cutting actuation wire 310 such that proximal retraction of the cutting actuation wire 310 actuates the cutting element 180, as described below.

While distal movement of the shaft 202 has been described as coupling the projection 234 of the connector 226 with the connector coupling portion 218 of the shaft 202 and then decoupling the coupler 250 of the slider from the slider coupling portion 216 and coupling the coupler 250 with the slider coupler receiver 278, it will be understood that the actuation assembly 200 may have other configurations and operations. For example, the distal movement of the shaft 202 relative to the spool 268 may couple the projection 234 of the connector 226 with the connector coupling portion 218, decouple the coupler 250 of the slider 248 from the slider coupling portion 216, and couple the coupler 250 with the slider coupler receiver 278 substantially simultaneously.

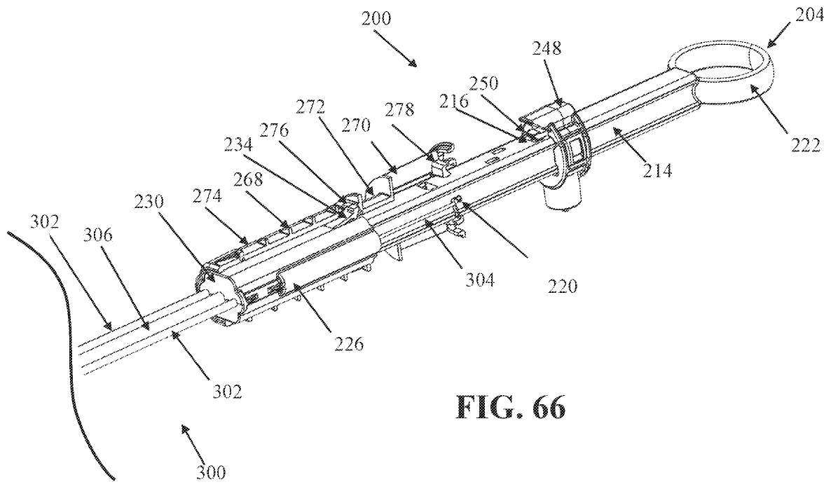

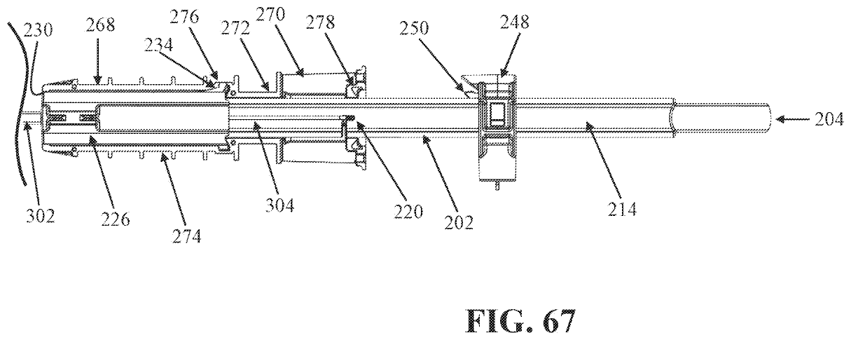

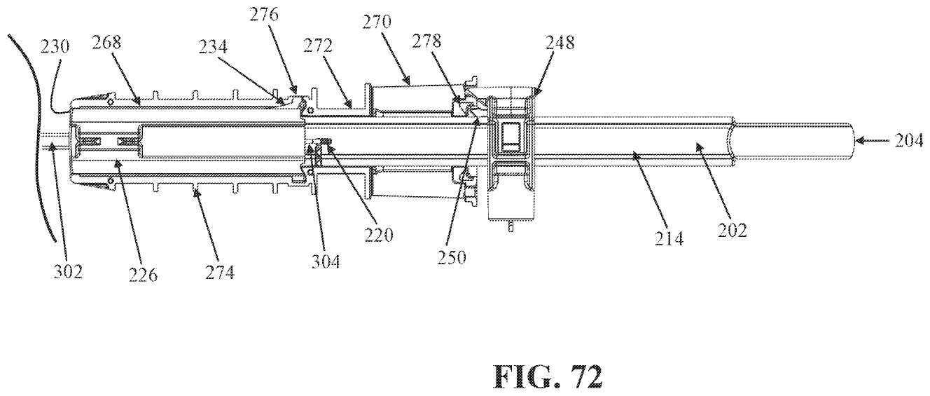

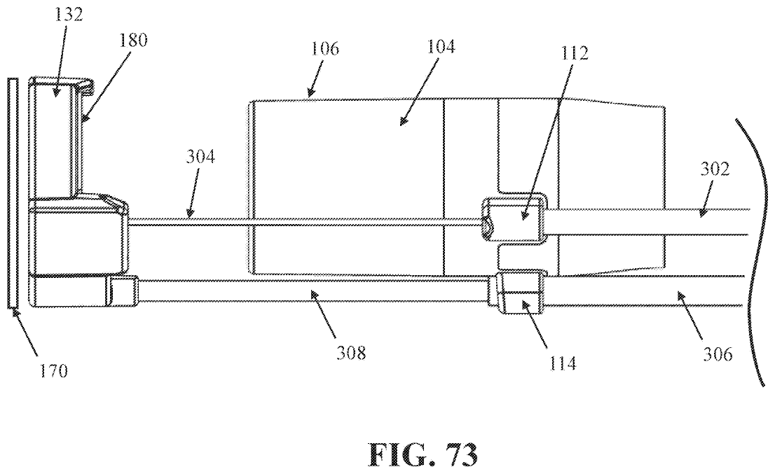

The coupled operation of the closure and retrieval assembly 102 and the actuation assembly 200 will now be described with reference to FIGS. 66-75. The catheter sheath assembly 300 may couple the closure and retrieval assembly 102 with the actuation assembly 200 such that actuation of the actuation assembly 200 may deploy and actuate the closure and retrieval assembly 102. The actuation assembly 200 may be coupled to the closure and retrieval assembly 102 via the catheter sheath assembly 300 to deploy the cinch element 170 from the housing 104 of the closure and retrieval assembly 102 to cinch tissue and to actuate the cutting element 180 to cut the target tissue. For example, the actuation assembly 200 may be coupled with the closure and retrieval assembly 102 to deploy the cinch element 170 and actuate the cutting element 180 with a single stroke of the shaft 202 of the actuation assembly 200.

The catheter sheath assembly 300 includes one or more clip deployment catheters 302, a deployment actuation wire 304 extending through each of the clip deployment catheters 302, one or more housing catheters 306, a collar catheter 308 extending through each of the housing catheters 306, and a cutting actuation wire 310 extending through each of the collar catheters 308. The catheter sheath assembly 300 may also a strain relief catheter 312 (FIG. 68) disposed around the housing catheter 306 and configured to reduce the stress and/or strain of the housing catheter 306. The catheter sheath assembly 300 may extend through a catheter deploying the endoscope to which the closure and retrieval assembly 102 is attached or may extend through a channel located within and extending through the endoscope. In the illustrated embodiment, the closure and retrieval device 100 includes two clip deployment catheters 302 and one housing catheter 306. However, it will be understood that the device 100 may include any suitable number of clip deployment catheters 302 and housing catheters 306.

As shown in FIGS. 66-70, the catheter sheath assembly 300 may couple the closure and retrieval assembly 102 and the actuation assembly 200 when the closure and retrieval assembly 102 is in the undeployed configuration and the actuation assembly 200 is in the unactuated configuration. The housing 104 may be disposed at the distal end of an endoscope with the collar 132 and cinch element 170 disposed around the outer surface 106 of the housing 104. The collar 132 may be disposed around a medial portion of the housing 104. The cinch element 170 may be disposed in the open position distally to the collar 132. The cutting element 180 may be disposed within the collar 132 and disposed around the outer surface 106 of the housing 104.

The connector 226 may be disposed at least partially beyond the distal end 206 of the shaft 202. The distal end 206 of the shaft 202 may be spaced apart from the shoulder 238 of the connector 226. The spool 268 may be disposed substantially around the connector 226. The projection 234 of the connector 226 may be coupled with or otherwise disposed in the projection receiving portion 276 of the spool 268. Optionally, the spool cover 284 may be disposed around the outside of the spool 268. The connector 226 and spool 268 may be slidable along a length of the shaft 202.

The slider 248 may be disposed toward the proximal end 204 of the shaft 202 with the coupler 250 of the slider 248 coupled with the slider coupling portion 216 of the shaft 202. The coupler 250 of the slider 248 may be spaced proximally apart from the proximal portion 270 of the spool 268. The slider 248 may be relatively fixed in place along the shaft 202.

The clip deployment catheters 302 and the housing catheter 306 may couple the housing 104 to the connector 226. The proximal end of the housing catheter 306 and the proximal ends of the clip deployment catheters 302 are coupled with the connector 226, such as with the distal end 230 of the connector 226. The distal ends of the clip deployment catheters 302 are coupled with the clip deployment catheter receivers 112 of the housing 104. Each clip deployment catheter 302 may be coupled to the clip deployment catheter receiver 112 via heat staking, adhesives, welding, over-molding, fasteners, or the like. The distal end of the housing catheter 306 is coupled with the catheter receiver 114 of the housing 104. The housing catheter 306 may be coupled with the catheter receiver 114 via heat staking, adhesives, welding, over-molding, fasteners, or the like.

The proximal end of each clip deployment catheter 302 may be disposed in one of the second bores 246 (FIG. 66) of the connector 226. In some embodiments, the proximal ends of the clip deployment catheters 302 are secured in one of the second bores 246 via heat staking, adhesives, welding, over-molding, fasteners, or the like. In other embodiments, the proximal end of each clip deployment catheter 302 extends proximally through one of the second bores 246 such that the proximal ends of the clip deployment catheters 302 are relatively unsecured.

The proximal end of the housing catheter 306 may be disposed in the first bore 240 of the connector 226, such as within the first and second portions 242, 244 of the first bore 240. The proximal end of the housing catheter 306 may be secured in the second portion 244 of the first bore 240 via heat staking, adhesives, welding, over-molding, fasteners, or the like. In embodiments including a strain relief catheter 312, the proximal end of the strain relief catheter 312 may be disposed in the first bore 240 of the connector 226, such as within the first portion 242 of the first bore 240. The proximal end of the strain relief catheter 312 may be secured in the first portion 242 of the first bore 240 via heat staking, adhesives, welding, over-molding, fasteners, or the like.

The position of the housing 104 may be relatively fixed to the position of the connector 226. For example, the clip deployment catheters 302 and the housing catheter 306 may be relatively inelastic such that housing 104 is held relatively in place when the connector 226 is maintained in position. The lengths of the clip deployment catheters 302 between the distal end 230 of the connector 226 and the clip deployment catheter receivers 112 may be substantially equivalent to or correspond with length of the housing catheter 306 between the distal end 230 of the connector 226 and the catheter receiver 114 such that the housing 104 remains aligned during deployment and actuation of the closure and retrieval assembly 102. Additionally, the clip deployment catheters 302 and the housing catheters 306 may be substantially inelastic in a longitudinal direction such that the clip deployment catheter receivers 112 and the catheter receiver 114 move in substantially together with movement of the connector 226.

In some embodiments, the clip deployment catheters 302 include a coating on an inner surface of the clip deployment catheter 302, on an outer surface of the clip deployment catheter 302, or both. The coating may decrease friction, such as to decrease friction between the outer surface of the clip deployment catheter 302 and the catheter sheath assembly or endoscope and/or to decrease friction between the inner surface of the clip deployment catheter 302 and the deployment actuation wire 304. The coating may be a hydrophilic material, PTFE, or parylene, or combinations thereof. Additionally or alternatively, the inner surface of the clip deployment catheters 302 may include a liner configured to decrease friction, such as a PTFE liner. In still further embodiments, the clip deployment catheters 302 each include a cover, such as a heat shrink, disposed around the outer surface of the clip deployment catheter 302, such as to increase the strength of the clip deployment catheter 302.

The deployment actuation wires 304 may couple the collar 132 to the shaft 202 of the actuation assembly 200. The deployment actuation wires 304 may be coupled with the collar 132 such that the collar 132 may move relative to the housing 104 via movement of the deployment actuation wires 304 through the clip deployment catheters 302. For example, the deployment actuation wires 304 may extend distally relative to the clip deployment catheters 302 when the shaft 202 is moved distally relative to the connector 226 such that the collar 132 moves distally relative to the housing 104. The collar catheter 308 may also be configured to operate with the shaft 202 of the actuation assembly 202 such that movement of the collar catheter 308 via movement of the shaft 202 also operates to move the collar 132 distally relative to the housing 104.

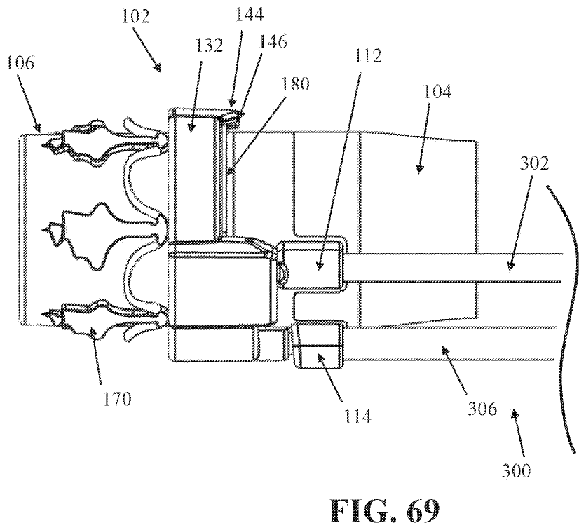

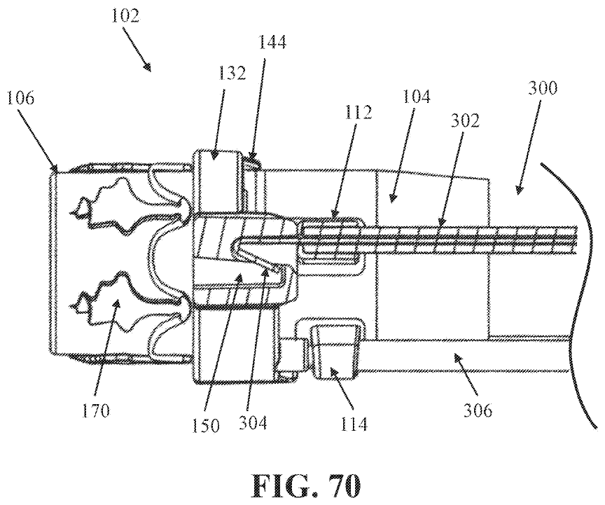

As shown in FIGS. 69-70, the distal end of each deployment actuation wire 304 is coupled to a proximal side of the collar 132, such as being coupled with one of the pockets 150 of the collar 132. Each deployment actuation wire 304 extends through one of the clip deployment catheters 302 and the respective clip deployment catheter receiver 112. The proximal portion of each deployment actuation wire 304 may extend through one of the second bores 246 and couple with the shaft 202. The proximal end of each deployment actuation wire 304 may be fixed relative to the shaft 202. The proximal ends of each of the deployment actuation wires 304 may be disposed in or otherwise coupled with the wire receiving portions 220 of the shaft 202. As shown in FIG. 68, the proximal end of each deployment actuation wire 304 may be bent into a substantially U-shape and received in one of the wire receiving portions 220 such that the deployment actuation wires 304 remain coupled with the shaft 202 and such that distal movement of the shaft 202 moves the deployment actuation wires 304 distally.

The distal ends of each of the deployment actuation wires 304 may be coupled with the collar 132 such that the position of the collar 132 is fixed relative to the distal ends of the deployment actuation wires 304. In one embodiment, as shown in FIG. 70, the distal end of each deployment actuation wire 304 is bent proximally in one of the pockets 150 of the collar 132 to couple the deployment actuation wire 304 with the collar 132. Each deployment actuation wire 304 may extend distally through one of the clip deployment catheters 302 and into the pocket 150. Each deployment actuation wire 304 may be inserted farther into the pocket 150 such that the distal end of the deployment actuation wire 304 bends and extends proximally in a V-shape. After the deployment actuation wires 304 are bent in the pocket 150, the distal end of the deployment actuation wire 304 may remain in the pocket 150 and the deployment actuation wire 304 may remain coupled with the collar 132.

While the distal end of the deployment actuation wires 304 have been described as being bent into a V-shape to couple the deployment actuation wire 304 with the collar 132, it will be understood that the distal ends of the deployment actuation wires 304 may be coupled with the collar 132 in other manners. For example, the distal ends of the deployment actuation wires 304 may be bent into a loop and secured around posts on the collar 132 and/or the distal ends of the deployment actuation wires 304 may be secured to the collar with adhesives, welding, fasteners, over-molding, or other similar methods.

The distal end of collar catheter 308 may be coupled with the collar 132 such that the collar 132 may be moved distally relative to the housing 104. The distal end of the collar catheter 308 is coupled with the collar 132, such as within the actuation passage 152 of the collar 132. The collar catheter 308 may be coupled with the collar 132 such that the collar catheter 308 may move in concert with the collar 132. The distal end of the collar catheter 308 may be coupled with the collar 132 via adhesives, welding, fasteners, over-molding, or other similar methods.

The proximal end of the collar catheter 308 may extend through the proximal end of housing catheter 306 and through the shaft receiving portion 232 of the connector 226. The collar catheter 308 may extend between the sides 214 of the bottom portion 212 of the shaft 202. The proximal end of the collar catheter 308 may abut a stop 215 of the shaft 202 (FIG. 68) which prevents the collar catheter 308 from moving proximally beyond the stop 215. The stop 215 may be one or more shoulders or projections extending perpendicularly from one or both of the sides 214 which may contact the proximal end of the collar catheter 308 and prevent the collar catheter 308 from moving farther proximally. The stop 215 may be sized, shaped, and configured such that the cutting actuation wire 310 may extend beyond the stop 215 relatively unrestricted.

The cutting actuation wire 310 may couple the cutting element 180 of the closure and retrieval assembly 102 to the slider 248 of the actuation assembly 200. The cutting actuation wire 310 may couple the cutting element 180 with the slider 248 such that proximal actuation of the slider 248 actuates the cutting element 180. The distal end of the cutting actuation wire 310 may extend proximally through the housing catheter 306 and the collar catheter 308 toward the actuation passage 152 of the collar 132. The distal end of the cutting actuation wire 310 may be coupled to the cutting element 180, such as to one or more legs 182 of the cutting element 180, such that proximal retraction of the cutting actuation wire 310 actuates the cutting element 180. For example, in embodiments where the cutting element 180 is a snare, proximal retraction of the cutting actuation wire 310 may cause the loop 184 to constrict, such as to cut target tissue. In some embodiments, the distal end of the cutting actuation wire 310 is welded to the cutting element 180. In other embodiments, the distal end of the cutting actuation wire 310 is coupled to the cutting element 180 via welding, fastening, over-molding, or other similar methods.

The proximal end of cutting actuation wire 310 may extend proximally through the connector 226 and along the shaft 202 toward the slider 248, such as between the sides 214 of the bottom portion 212 of the shaft 202. The proximal end of the cutting actuation wire 310 may be coupled with the slider 248, such as within the actuation wire receiving portion 254 of the slider 248. The proximal end of the cutting actuation wire 310 may be secured in the actuation wire receiving portion 254 of the slider 248 via adhesives, a fastener, welding, over-molding, or the like, or combinations thereof.

While not shown, tissue may be recruited into the tissue receiving portion 110 of the housing 104 when the closure and retrieval assembly 102 is in the undeployed configuration and the actuation assembly 200 is in the unactuated configuration. Tissue may be recruited into the housing via a tissue recruiting means, such as a suction, a grasper, or other means, implemented from a channel located within and extending through an endoscope as described above. Additionally, the target tissue may be marked by one or more markers deployed though a channel located within and extending through the endoscope.

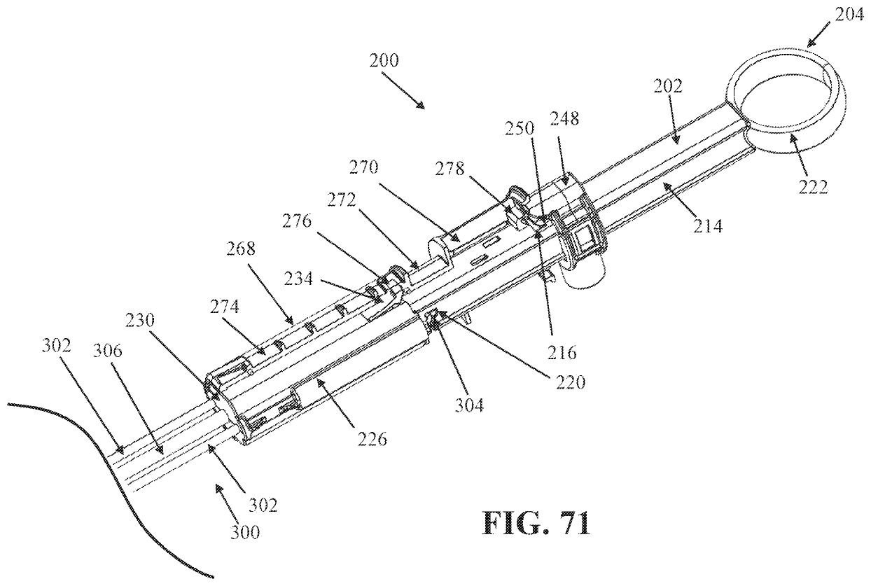

As shown in FIGS. 71-73, the actuation assembly 200 may be moved to the partially actuated position to move the closure and retrieval assembly 102 to the deployed configuration. The spool 268 may be held relatively stationary and the shaft 202 may be moved distally through the spool 268 and the connector 226. For example, a user may hold the spool 268 in place in one hand and move the shaft 202, such as via the ring 222, and push the shaft 202 toward the spool 268 with the other hand.

The shaft 202 may be moved distally until the distal end 206 of the shaft 202 abuts the shoulder 238 of the connector 226 and/or the projection 234 of the connector 226 flexes downwardly to couple with the connector coupling portion 218 of the shaft 202. The spool 268 and the connector 226 may decouple when the projection 234 of the connector 226 flexes downwardly to couple the connector 226 with the shaft 202. The spool 268 may then move proximally relative to the shaft 202 and the connector 226. The slider 248 may be disposed near the proximal portion 270 of the spool 268.

As the connector 226 is initially held relatively stationary with the spool 268, the clip deployment catheters 302 and the housing catheter 306 may remain in position coupled to the distal end 230 of the connector 226, thereby keeping the housing 104 in position, such as above target tissue. The proximal ends of the deployment actuation wires 304 are connected to the wire receiving portions 220 of the shaft 202 and are moved distally relative to the clip deployment catheters 302 as the shaft 202 is moved distally relative to the connector 226. The proximal end of collar catheter 308 may abut the stop 215 of the shaft 202 such that the collar catheter 308 is moved distally through the housing catheter 306 as the shaft 202 is moved distally.

The distal movement of the deployment actuation wires 304 relative to the clip deployment catheters 302 and/or the distal movement of the collar catheter 308 relative to the housing catheter 306 may move the collar 132 distally relative to the housing 104. The distal movement of the collar 132 may abut the proximal end of the cinch element 170 such that the cinch element 170 is moved distally relative to the housing 104. The distal movement of the collar 132 may move the cinch element 170 distally off of the housing 104. When the cinch element 170 is moved off of the housing 104, the cinch element 170 may move to the closed position thereby cinching the target tissue.

Continued distal movement of the deployment actuation wires 304 and/or the collar catheter 308 may cause the collar 132 to be moved distally past the distal end of the housing 104. The inner surface 134 of the collar 132 may be less than the outer diameter of the cinch element 170 in the closed position such that the collar 132 is not moved distally past the cinch element 170. In the deployed configuration, the collar 132 may be disposed such that the cutting element 180 is at the desired position relative to the cinch element 170. The components of the closure and retrieval assembly 102 and the components of the actuation assembly 200 may be sized, shaped, and configured such that the cutting element 180 is disposed in the desired position relative to the cinch element 170 in the closed position. For example, the starting position of the actuation assembly 200, the size of the shaft receiving portion 232 of the connector 226, and/or the position and configuration of the wire receiving portions 220 may be selected such that the collar 132 is moved distally to the desired position when the closure and retrieval assembly 102 is in the deployed configuration. The ring portion 138 of the collar 132 may be sized, shaped, and configured such that the cutting element 180 is disposed in the desired position when the distal end of the ring portion 138 abuts the cinch element 170. Additionally or alternatively, the cinch element 170 may have a thickened proximal portion in the closed position that positions the cutting element 180 in the desired position. In some embodiments, when the closure and retrieval assembly 102 is in the fully deployed configuration, the cutting element 180 is disposed between about 1 mm and about 6 mm, such as between about 2 mm and about 5 mm, such as about 4 mm, proximally to the where the cinch element 170 cinches tissue.

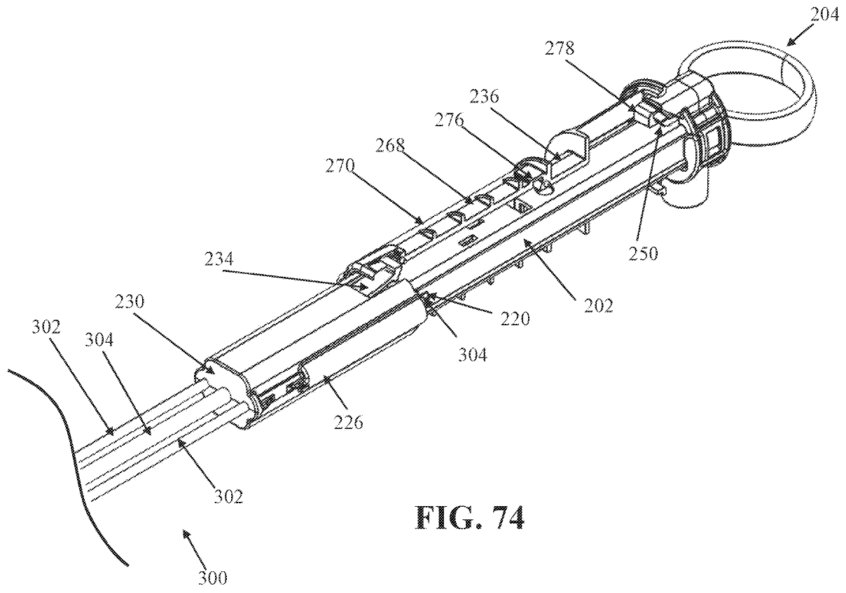

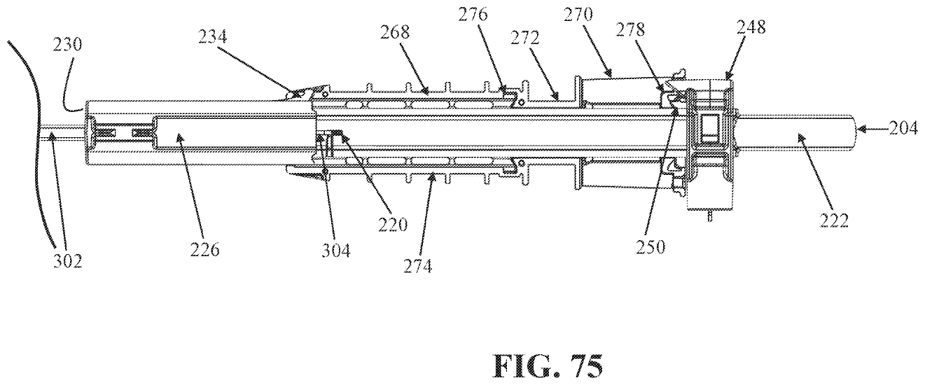

As shown in FIGS. 74-75, the actuation assembly 200 may be moved to the fully actuated position to actuate the cutting element 180 of the closure and retrieval assembly 102. The shaft 202 may be moved farther distally relative to the spool 268 and/or the spool 268 may be moved proximally relative to the shaft 202 and the connector 226. The relative proximal movement of the spool 268 may decouple the coupler 250 of the slider 248 from the slider coupling portion 216 of the shaft 202. Continued relative proximal movement of the spool 268 may couple the coupler 250 of the slider 248 with the slider coupler receiver 278 of the spool 268 such that the slider 248 moves in concert with the spool 268.

Continued relative proximal movement of the spool 268 may move the slider 248 proximally farther away from the connector 226 and the wire receiving portions 220 of the shaft 202. The proximal end of the cutting actuation wire 310 may be coupled with the actuation wire receiving portion 254 of the slider 248 such that proximal movement of the slider 248 proximally moves the proximal end of the cutting actuation wire 310. As the deployment actuation wires 304 are coupled with the wire receiving portions 220 of the shaft 202, the proximal end of the collar catheter 308 abuts the stop, the proximal ends of the housing catheter 306 and the clip deployment catheters 302 are coupled with the connector 226, and the connector 226 and the shaft 202 are relatively stationary, the collar 132 and the housing 104 may remain relatively in place. The relative proximal movement of the cutting actuation wire 310, such as in relation to the relatively stationary housing 104, collar 132, and deployment actuation wires 304, may actuate the cutting element 180. The actuation of the cutting element 180 may cut the target tissue at the desired position, such as via any manner described above.

In some embodiments, the cutting element 180 may be energized to assist in cutting or otherwise severing the target tissue. The energy source 286 may provide energy to the energy connector 266 of the slider 248, which may then be directed to the cutting actuation wire 310 and to the cutting element 180, such that the cutting element 180 is energized to cut the target tissue. In some embodiments, a user may activate the switch 288 during actuation of the actuation assembly 200 to supply energy from the energy source 286 to the cutting element 180.

After the target tissue is cut, the closure and retrieval assembly 102 may be removed from the body. The target tissue may continue to be recruited into the tissue receiving portion 110 of the housing 104 such that the cut target tissue is removed with the closure and retrieval assembly 102. Alternatively, the cut target tissue may first be removed from the body via a channel located within and extending through the endoscope. In some embodiments, the collar 132 may be retracted proximally such that the collar 132 is disposed around the outer surface 106 of the housing 104 before retraction. The collar 132 may be retracted proximally by moving the shaft 202 proximally relative to the spool 268 in the opposite of the deployment actuation. Retracting the collar 132 to a position around the housing 104 before removal of the closure and retrieval assembly 104 before extraction may reduce trauma in the removal process.

One of ordinary skill in the art will now appreciate that the present invention provides a closure and retrieval device configured to deploy a cinch element and remove target tissue, such as with a single stroke of an actuation assembly. Although the present invention has been shown and described with reference to particular embodiments, equivalent alterations and modifications will occur to those skilled in the art upon reading and understanding this specification. The present invention includes all such equivalent alterations and modifications and is limited only by the scope of the following claims in light of their full scope of equivalents.

While various inventive aspects, concepts and features of the disclosures can be described and illustrated herein as embodied in combination in the example embodiments, these various aspects, concepts, and features can be used in many alternative embodiments, either individually or in various combinations and sub-combinations thereof. Unless expressly excluded herein all such combinations and sub-combinations are intended to be within the scope of the present application. Still further, while various alternative embodiments as to the various aspects, concepts, and features of the disclosures-such as alternative materials, structures, configurations, methods, devices, and components, alternatives as to form, fit, and function, and so on-can be described herein, such descriptions are not intended to be a complete or exhaustive list of available alternative embodiments, whether presently known or later developed. Those skilled in the art may readily adopt one or more of the inventive aspects, concepts, or features into additional embodiments and uses within the scope of the present application even if such embodiments are not expressly disclosed herein.

Additionally, even though some features, concepts, or aspects of the disclosures can be described herein as being a preferred or exemplary arrangement or method, such description is not intended to suggest that such feature is required or necessary unless expressly so stated. Still further, example or representative values and ranges can be included to assist in understanding the present application, however, such values and ranges are not to be construed in a limiting sense and are intended to be critical values or ranges only if so expressly stated.

Moreover, while various aspects, features and concepts can be expressly identified herein as being inventive or forming part of a disclosure, such identification is not intended to be exclusive, but rather there can be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. The words used in the claims have their full ordinary meanings and are not limited in any way by the description of the embodiments in the specification.

1. A closure and retrieval assembly for an endoscopic device, the closure and retrieval assembly comprising:
a housing having an outer surface and an inner surface defining a tissue receiving portion;
a collar having an inner surface and an outer surface, the inner surface being configured to fit around the outer surface of the housing;
a cinch element movable between an open position configured to fit around the outer surface of the housing and a closed position configured to cinch tissue recruited into the tissue receiving portion; and
a cutting element coupled with the collar;
wherein the closure and retrieval assembly is movable between an undeployed configuration and a deployed configuration;
wherein the collar and the cinch element are disposed around the outer surface of the housing when the assembly is in the undeployed configuration; and
wherein the cinch element and the collar are disposed distally relative to the housing when the assembly is in the deployed configuration.

2. The closure and retrieval assembly of claim 1, wherein the cutting element is operably configured to cut tissue when the closure and retrieval assembly is in the deployed configuration.

3. The closure and retrieval assembly of claim 1, wherein the collar includes an extension with a flange configured to retain the cutting element in the collar during deployment.

4. The closure and retrieval assembly of claim 1, wherein distal movement of the collar relative to the housing deploys the cinch element.

5. The closure and retrieval assembly of claim 1, wherein the cutting element is a snare.

6. The closure and retrieval assembly of claim 1, wherein an outer diameter of the cinch element is larger than a diameter of the inner surface of the collar.

7. An actuation assembly for an endoscopic device, the actuation assembly comprising:
a shaft having a slider coupling portion and a connector coupling portion;
a slider having a coupler configured to operatively couple with the slider coupling portion of the shaft;
a connector having a projection configured to operative couple with the connector coupling portion of the shaft; and
a spool configured to slide along the shaft and configured to operatively couple with the connector and the slider;
wherein the actuation assembly is movable between an unactuated configuration with the connector and the spool disposed near a distal end of the shaft, a partially actuated configuration, and a fully actuated configuration with the spool and the slider disposed near a proximal end of the shaft;
wherein the connector is coupled with the spool and the slider is coupled with the shaft when the assembly is moved from the unactuated configuration to the partially actuated configuration;
wherein the slider is coupled with the spool and the connector is coupled with the shaft when the assembly is moved from the partially actuated configuration to the fully actuated configuration.

8. The actuation assembly of claim 7, wherein the spool includes a projection receiving portion configured to operatively couple with the projection of the connector.

9. The actuation assembly of claim 7, wherein the spool is configured to slide over the connector when the projection of the connector is coupled with the connector coupling portion of the shaft.

10. The actuation assembly of claim 7, wherein the spool includes slider coupler receiver configured to operatively couple with the coupler of the slider.

11. The actuation assembly of claim 7, wherein the slider coupler receiver is configured to operatively decouple the coupler of the slider from the slider coupling portion.

12. The actuation assembly of claim 7, wherein the slider includes a connector configured to receive energy from an energy source.

13. The actuation assembly of claim 7, wherein the connector includes a shoulder configured to abut the distal end of the shaft when the assembly is in the partially actuated configuration.

14. The actuation assembly of claim 7, further comprising a spool cover configured to retain the spool around the shaft.

15. A closure and retrieval device comprising:
a closure and retrieval assembly movable between an undeployed configuration and a deployed configuration, the closure and retrieval assembly comprising:
a housing having an outer surface and an inner surface defining a tissue receiving portion;
a collar including a cutting element, the collar being disposed around the outer surface of the housing in the undeployed configuration; and
a cinch element disposed around the outer surface of the collar and distal to the collar in the undeployed configuration;
an actuation assembly movable between an unactuated configuration and an actuated configuration, the actuation assembly comprising:
a shaft extending between a proximal end and a distal end;
a connector proximally slidable relative to the shaft in the undeployed configuration and coupled to the shaft in the actuated configuration;
a slider coupled to the shaft in the unactuated configuration and proximally slidable relative to the shaft in the actuated configuration; and
a spool configured to slide along the shaft and configured to operatively couple with the connector and the slider; and
a catheter sheath assembly configured to operatively couple the closure and retrieval assembly and the actuation assembly;
wherein distal movement of the shaft relative to the spool is configured to move the connector proximally relative to the shaft until the connector couples with the shaft and the spool decouples the slider from the shaft such that the slider is slidable proximally relative to the shaft;
wherein actuation of the actuation assembly is configured to move the closure and retrieval assembly from the undeployed configuration to the deployed configuration; and
wherein the cinch element is moved distally from the housing when the closure and retrieval assembly is moved from the undeployed configuration to the deployed configuration.

16. The closure and retrieval device of claim 15, wherein the catheter sheath assembly includes a cutting actuation wire coupled between the slider and the cutting element and configured to actuate the cutting element when the closure and retrieval assembly is in the deployed configuration.

17. The closure and retrieval device of claim 15, wherein the catheter sheath assembly couples the closure and retrieval assembly with the actuation assembly such that proximal movement of the connector relative to the shaft deploys the cinch element and the collar from the housing.

18. The closure and retrieval device of claim 15, wherein the cinch element is disposed around the housing in an open position and moves to a closed position when deployed from the housing.

19. The closure and retrieval device of claim 15, wherein the cutting element is configured to cut tissue proximally to the deployed cinch element when the closure and retrieval device is in the deployed configuration.

20. The closure and retrieval device of claim 15, wherein the catheter sheath assembly includes a deployment actuation wire coupling the collar and the shaft such that distal movement of the shaft moves the collar distally relative to the housing.
